SEQUENCE LISTING (10 sequences)

SEQ ID NO: 1 - protein, 96 aa, Homo sapiens
GIIAAYQNPASWKSMKVTVAFNQFGPDIFASKNFHLQKNKLTSGKIASCLNYGLVHVANNNYDPSGKYEGGNIYTKKEAFNVEQFAKLTGFTLMGR

SEQ ID NO: 2 - protein, 192 aa, Homo sapiens
GIIAAYQNPASWKSMKVTVAFNQFGPDIFASKNFHLQKNKLTSGKIASCLNYGLVHVANNNYDPSGKYEGGNIYTKKEAFNVEQFAKLTGFTLMGRGIIAAYQNPASWKSMKVTVAFNQFGPDIFASKNFHLQKNKLTSGKIASCLNYGLVHVANNNYDPSGKYEGGNIYTKKEAFNVEQFAKLTGFTLMGR

SEQ ID NO: 3 - protein, 24 aa, Oryza sativaL. cv Manngetsumochi
MASSVFSRFSIYFCVLLLCHGSMA

SEQ ID NO: 4 - protein, 24 aa, Oryza sativaL. cv Manngetsumochi
MASINRPIVFFTVCLFLLCDGSLA

SEQ ID NO: 5 - protein, 23 aa, Oryza sativaL. cv Manngetsumochi
MASKVVFFAAALMAAMVAISGAQ

SEQ ID NO: 6 - DNA, 3350 bp, Artificial Sequence
  Description of Artificial Sequence Artificially constructed DNA sequence
  CDS (2333)..(2713)
acagattcttgctaccaacaacttcacaaagtagtagtcaaccaaaactatgctaaggaatcacctcacttccgcccatgaccgtgagcacgactgttcaaacagtttgttaatctctacaaagaaggtacactttacctacacaacgccactaacctgagttacccagcccatgcaaaatagccacgtcttgtgacttaagggatttcgcgacaaggcatttcgaaagcccacacaaggacaccttatgaaaactggaggggtcccacagaccaacaacaagttaggtcccaaaccatgttgtgccaggaaaaatccaaggggtcctccccaacaccaccccgacaaatccacttgtccattggcatcaagatttgcctgacctagctaattactcagccaggcatgtcacaattcacccatgtggtcacacatgttatggttggatgaaattctaaaggaatcggtccatatgagcaagaccgagaaaccataccaccagtacttctaccgaaatacgagtttagtaaactcatttgttttcaaggcacccgacccaggtgtgtcgggttttccagggattttgtaaacccaagttttacccatagttgatcattcaaattttgaggagggtcattggtatccgtacctgagggcacgaatactgagacctagcattgtagtcgaccaaggaggttaatgcagcaattgtaggtggggcctgttggttatattgcaaactgcggccaacatttcatgtgtaatttagagatgtgcattttgagaaatgaaatacttagtttcaaattatgggctcaaaataatcaaaggtgacctaccttgcttgatatcttgagcttcttcctcgtattccgcgcactaggactcttctggctccgaagctacacgtggaacgagataactcaacaaaacgaccaaggaaaagctcgtattagtgagtactaagtgtgccactgaatagatctcgatttttgaggaattttagaagttgaacagagtcaatcgaacagacagttgaagagatatggattttctaagattaattgattctctgtataaagaaaaaaagtattattgaattaaatggaaaaagaaaaaggaaaaaggggatggcttctgctttttgggctgaaggcggcgtgtggccagcgtgctgcgtgcggacagcgagcgaacacacgacggagcagctacgacgaacgggggaccgagtggaccggacgaggatgtggcctaggacgagtgcacaaggctagtggactcggtccccgcgcggtatcccgagtggtccactgtctgcaaacacgattcacatagagcgggcagacgcgggagccgtcctaggtgcaccggaagcaaatccgtcgcctgggtggatttgagtgacacggcccacgtgtagcctcacagctctccgtggtcagatgtgtaaaattatcataatatgtgtttttcaaatagttaaataatatatataggcaagttatatgggtcaataagcagtaaaaaggcttatgacatggtaaaattacttacaccaatatgccttactgtctgatatattttacatgacaacaaagttacaagtacgtcatttaaaaatacaagttacttatcaattgtagtgtatcaagtaaatgacaacaaacctacaaatttgctattttgaaggaacacttaaaaaaatcaataggcaagttatatagtcaataaactgcaagaaggcttatgacatggaaaaattacatacaccaatatgctttattgtccggtatattttacaagacaacaaagttataagtatgtcatttaaaaatacaagttacttatcaattgtcaagtaaatgaaaacaaacctacaaatttgttattttgaaggaacacctaaattatcaaatatagcttgctacgcaaaatgacaacatgcttacaagttattatcatcttaaagttagactcatcttctcaagcataagagctttatggtgcaaaaacaaatataatgacaaggcaaagatacatacatattaagagtatggacagacatttctttaacaaactccatttgtattactccaaaagcaccagaagtttgtcatggctgagtcatgaaatgtatagttcaatcttgcaaagttgcctttccttttgtactgtgttttaacactacaagccatatattgtctgtacgtgcaacaaactatatcaccatgtatcccaagatgcttttttattgctatataaactagcttggtctgtctttgaactcacatcaattagcttaagtttccataagcaagtacaaatagctatggcgagttccggtttctctcggttttctatatacttttgtgttcttctattatgccacggttctatggcccagcccatgggcatcatcgcagcttaccaaaatccagcaagctggaagagtatgaaggttacagttgcattcaaccaattcggtcctgatatctttgctagcaagaatttccacctccagaaaaataagctcacaagtggcaagattgcaagctgcttgaactatggattggttcatgtagctaacaataactatgatccaagcggtaagtatgagggtggcaacatctacactaagaaggaagcattcaacgtagagcaatttgcaaagctcacaggcttcactctcatgggacgcaaggacgagttgaagagctctgtaattgagaactagtatcggcgtagagtaaaataaaacaccacaagtatgacacttggtggtgattctgttcgatatcagtactaaataaaggttacaaacttcttaattttcctacttcatgccatggatattccattatggactatagtggacagggccggtctatgattttgagggccctaggaactcatcgcgatgggcctcaagctatatataaaatttattgatatatatagacgctaattttacttgcaaaatgaaaacaaatacatctatatattaaatttaacattcctggtaattatcaagaaataaaatcgaccaaaataacaatatatttgtaacttggaactaatataattatttattaacttaatgaagaatagaaccccgtcatatccattgcttcctatgaaaagatacttcttcgggtatttcttgatgcaaaatcataaagaacggtattaagatcaatagtgtccaagatatccttctcgattgagcacatagccaagccatttaaccttatttgcgacagttgatctcaaatagtttttcaacaacttcaattttgataaacttatttcagctgaagctaccatcataggtaaagttaagagaattc

SEQ ID NO: 7 - protein, 127 aa, Artificial Sequence
  Description of Artificial Sequence  Putative amino acid sequence coded by artificially constructed DNA sequence
MASSGFSRFSIYFCVLLLCHGSMAQPMGIIAAYQNPASWKSMKVTVAFNQFGPDIFASKNFHLQKNKLTSGKIASCLNYGLVHVANNNYDPSGKYEGGNIYTKKEAFNVEQFAKLTGFTLMGRKDEL

SEQ ID NO: 8 - DNA, 1474 bp, Oryza sativa
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagct

SEQ ID NO: 9 - DNA, 824 bp, Oryza sativa
actggataattataatatcagttaaaattgaaaataatgcaacttcatacttgcatggtgtcagtagtgcctgcctaagaaatgtgtcttgtcataatatgattacatgaaatatgtttacttcctcgtttctctttatttgtaagataaagaactagatatgtggaaagtaggatagcaaagagtatggccaaactctaatctttgctttattttttgggatggacccaaaatttgtttctcctttacttctttccctttacaacaatgttctttacttccaattcttattaacaaaactccaaatacatgccaaactgcatatgtatgtatgctattaaggcacatttacaaagctccaagtttacctactcaatcattcacatatggcgatgactcaaactcttaattgttatctggtaagctgtgacttgtgtaacacattctacaagtcccatacgaattctgttcacaaaagtttctttgtccagctcataatttacaaaactgcaaaatgccaaagcaatctggcacaaccttatcatcatattttctttccacgcattaaagcactggcagaattatctttgtgtagatattccaaaagtattggttgaataaatgtccaaataaattccatgcctcatgatttccagcttatgtggcctccactaggtggttttgcaaaggccaaactctttcctggcttacacagctaccagcatgtataaataggcccctaggcaaccattattccatcatcctcaacaatattgtctacaccatctggaatcttgtttaacactagtattgtagaatcagca

SEQ ID NO: 10 - DNA, 931 bp, Oryza sativa
gatcttttaaccgtgctacgctgggttaattagcgatggtgcaggtcacgtacccaaatttcttcactgttggatcaactagagtagttaaacgagggcatgtgatgaaggctagctatttgaaattttccaattatccctgcataagtcaggctacaatagcacctggactacatgcagggattacaaaataggtggtaaccacatttaccgcgttaaccctatcaaattcaaataaattttaaaagtaatttgatttttttaataaattttgtatggtttctcaagctttattttggttaccgtgcttactgcggaggcaatgggaaaccctcactagaagttgcacctgttcttgtctgtgcaccatatcatgttgaatcatgtgcgttgtgtctttcggaagaaccgatttactacatgactcatcaattccactttacgtatcaaaaggtttgttatgggggcaatgcttttgtgaaattaaatttttattttgcgtcacgttgtatctagttaaacactacctacctaccattacaaaacctcattccacaaaacgatgcatctagataaaaaatatgacatgtaaagtgagtaatgactcatgtttattatcaaaaatcgataacaatcaaatgatataggtagtaaagtacctttgaaatggcatgtccaagtatgtgtagctccacctagcacaatatcccaagtgatcatcataaaaggcatacaaatacaagcagccgatgatgcacacaagaaacaacacaaattgcacaaaaccaaaagcaaccgatgccttgagcatagagatcatgctattcccactataaatacaaatgcaccatatcaagatgctcctcacccttactgaaaaatcacaaacatcaaaacgttataagagttctctagcatccatcacatagcc

(19) United States
(12) Patent Application Publication
(10) Pub. No.: US 2007/0136896 A1
(43) Pub. Date: Jun. 14, 2007

(54) Method of accumulating allergen-specific t cell antigen determinant in plant and plant having the antigen determinant accumulated therein

(76) Inventors: Fumio Takaiwa, Tsukuba-shi (JP); Hidenori Takagi, Tsukuba-shi (JP)

Correspondence Address: Michael L Goldman;Nixon Peabody, Clinton Square, P O Box 31051, Rochester, NY, 14603-1051, US

(21) Appl. No.: 10/554,308

(22) PCT Filed: Apr. 23, 2004
(86) PCT No.: PCT/JP04/05938

§ 371 Date: Apr. 17, 2006

(30) Foreign Application Priority Data
Apr. 24, 2003 (JP) 2003-120639

(51) Int. Cl. A01H 1/00; C12N 15/82; C12N 5/04; C07K 14/705; C07K 14/415

(52) U.S. Cl. 800/288; 530/350; 435/419; 435/468

(57) ABSTRACT

Disclosed herein are techniques for accumulating a human T-cell epitope in rice albumen, particularly a method of directly accumulating a T-cell epitope-linked peptide, such as 7 Crp, in rice seeds and a method of inserting 7 Crp into a variable region of glutelin, the major storage protein of rice, to express and accumulate 7 Crp as a part of the glutelin storage protein. Rice producing the T-cell epitope-linked peptide developed in accordance with the present invention is expected to function as an edible vaccine against Japanese cedar pollinosis.

### TECHNICAL FIELD

The present invention relates to methods of accumulating an allergen-specific T-cell epitope in a plant, and plants having the epitope accumulated therein.

### BACKGROUND ART

In recent years, a radical treatment for allergic disease has been a hyposensitization therapy in which an allergen per se is administered by a conventional injection at stepwise increased doses over a long period of time so as to decrease allergen-specific immunoreactions. However, it has been noted that the allergens used in this therapy retain reactivity with the IgE antibody bound to mast cells which cause allergic symptoms and, therefore, may result in problematic side effects, such as anaphylactic shock.

Recently, a peptide immunotherapy involving the administration of an allergen-derived T-cell epitope peptide has drawn much attention. The action mechanism thereof is presumed to involve the induction of unresponsiveness or deletion of allergen-specific type 2 helper T cells. T-cell epitope peptide immunotherapy is quite safe because it generally involves neither B cell epitopes, which cause allergic reactions, nor binding to the allergen-specific IgE antibody; as a result, it minimizes the harmful side effects observed in conventional hyposensitization therapy.

T-cell epitope peptides derived from Japanese cedar pollinosis allergen show a high reactivity toward specific T cells, suggesting a capability thereof to induce T-cell unresponsiveness through oral administration (see, e.g., Nonpatent references 1 to 4). Although, from these facts, T-cell epitope peptide has been expected to be used as a peptide vaccine for the treatment of Japanese cedar pollinosis, an actual application form has remained undeveloped.

To date, a method for actually accumulating an allergen-specific T-cell epitope in a useful plant or a useful plant comprising such an epitope accumulated therein has been hitherto unknown.
[Nonpatent reference 1] Kazuki Hirahara et al., J. Allergy Clin. Immunol., Vol. 102, p. 961-967, 1998.
[Nonpatent reference 2] Kazuki Hirahara et al., J. Allergy Clin. Immunol., Vol. 108, p. 94-100, 2001.
[Nonpatent reference 3] Toshio Sone et al., J. Immunology, p. 448-457, 1998.
[Nonpatent reference 4] Tomomi Yoshitomi, et al., Immunology, Vol. 107, p. 517-522, 2002.

### DISCLOSURE OF THE INVENTION

The present invention has been made in view of such circumstances, and an objective thereof is to develop a plant comprising a T-cell epitope peptide accumulated therein so as to apply the T-cell epitope as a peptide vaccine for the treatment or prevention of Japanese cedar pollinosis. More specifically, an objective of the present invention is to provide a method for accumulating an allergen-specific T-cell epitope in a plant and a plant comprising the epitope accumulated therein.

The present inventors conducted exhaustive studies to achieve the above-described objectives. Specifically, the present inventors succeeded in developing rice plants that have a T-cell epitope peptide accumulated therein by producing an artificial gene encoding a hybrid peptide (7 Crp) in which seven human major T-cell epitopes comprising 12 to 19 amino acid residues observed in Japanese cedar pollen allergens Cry j1 and Cry j2 presented by antigen-presenting cells (macrophages) are linked, and expressing this gene specifically in albumen, the edible part of rice.

Accordingly, it is possible to radically treat Japanese cedar pollinosis by eating the rice of the present invention (oral administration) so as to induce unresponsiveness or deletion of Japanese cedar pollen allergen-specific type 2 helper T cells through the immune tolerance mechanism.

Methods of accumulating the human T-cell epitope in rice albumen were developed in the present invention: namely, a method for directly accumulating 7 Crp peptide in seeds; and a method for accumulating it by inserting into a variable region of glutelin, the major storage protein of rice, to express it as a part of the glutelin storage protein. Using the techniques of the present invention, a peptide comprising the sequentially linked seven T-cell epitopes (7 Crp) was successfully accumulated in rice seeds at a high level. In those seeds, a maximum of 60 μg of the T-cell epitope-linked peptide per seed was accumulated, corresponding to about 4% of the total protein contained in the seed.

When an allergen epitope peptide is accumulated in such an edible part at a high level, it becomes possible to treat allergic reactions derived from such allergens by oral intake thereof through the immune tolerance mechanism.

Previously, studies have reported studies that the oral administration of mouse T-cell epitope-linked peptides to mice, at dosages of 40 to 200 μg or 2.5 to 250 μg of the peptide, results in the induction of immune tolerance (Hirahara et al. Oral administration of a dominant T-cell determinant peptide inhibits allergen-specific TH1 and TH2 cell responses in Cry j2-primed mice J. Allergy Clin. Immunol. 1998 102, 961-967; Yoshitomi et al. Immunology 2002, 107, 517-522). When applying these results to humans, presuming that the mouse body weight is 20 g and the human body weight is 60 kg, the amount of T-cell epitope-linked peptide necessary to induce immune tolerance in human is estimated to be 7.5 mg (2.5 μg) to 250 mg. Noting that 30 μg of the peptide is accumulated per seed and the weight of one seed is about 20 mg, this estimated necessary amount correlates to a requirement of 3.75 mg to 375 mg of seeds producing T-cell epitope-linked peptide. Accordingly, when converted based on that seed amount, 2.5 g to 250 g, an amount which can be sufficiently supplied by a daily diet intake of 100 g to 150 g, should be sufficient to induce immune tolerance. Therefore, the rice developed in accordance with the present invention, producing a T-cell epitope-linked peptide, is strongly expected to function as an edible vaccine against Japanese cedar pollinosis.

Further, since the 7 Crp peptide accumulated by the method of the present invention differs in its accumulation site in albumen, its induction mechanism of immune tolerance through the mucosal immune system is expected to be somewhat different.

No alteration in the T-cell epitope-linked peptide was observed before and after a 20-min heat treatment of transformant seeds of the present invention. Accordingly, the T-cell epitope-linked peptide is stably present even after cooking rice. Further, no significant changes in the major allergenic proteins of rice were observed; also, no sugar chain, which may possibly cause an allergic reaction, was found to be bound to the T-cell epitope-linked peptide. Thus, the studies performed to date have found no risks concerning the function and safety of the T-cell epitope-linked peptide of the instant invention as an edible vaccine against pollinosis.

Further, as compared to conventional production of peptide proteins, involving culturing E. coli or the like, the present invention can produce recombinant peptides more economically. That is, in the case of rice, one seed can produce 1000 to 2000 seeds. In addition, production with seeds enables the oral intake thereof as they are, without purification. Peptides produced in seeds are extremely stable and free from decomposition and activity loss, even when seeds are left at standing at room temperature for one year or more. Further, as compared to tank culture, production by seeds allows for easy yield control, for example, by adjusting the number of seeds to be planted.

As described above, by the method developed by the present inventors, useful rice plants comprising T-cell epitopes highly accumulated in albumen were successfilly produced for the first time. Further, the present inventors found out that the “rice comprising a T-cell epitope accumulated in albumen” produced by the method of the present invention is effective in actually inducing immune tolerance, as demonstrated by the efficacy assessment test-using mice. That is, the present invention conclusively demonstrates that the rice of the present invention can enable the mitigation of pollinosis.

As described above, the present inventors succeeded in developing methods for accumulating an allergen-specific T-cell epitope in a plant, as well as plants comprising the epitope accumulated therein (for example, rice plants having an allergen-specific T-cell epitope highly accumulated in seed albumen), and thus completed the present invention. More specifically, the present invention provides the following:
[1] a DNA comprising a structure in which a DNA according to any one of the following (a) to (c) is placed under the control of a storage protein promoter,
   (a) a DNA made by adding a DNA encoding a storage protein signal sequence to the 5′-end of a DNA encoding an allergen-specific (allergen-derived) T-cell epitope peptide, and/or a DNA encoding an endoplasmic reticulum (ER)-retention signal sequence to the 3′-end thereof;
   (b) a DNA encoding a polypeptide in which a storage protein signal sequence is added to the N-terminal of an allergen-specific T-cell epitope peptide, and/or an ER-retention signal sequence to the C-terminal thereof; and
   (c) a DNA encoding a polypeptide having a structure in which an allergen-specific T-cell epitope peptide is inserted into a variable region of a storage protein;
[2] the DNA according to [1], wherein the T-cell epitope peptide comprises at least two, preferably seven, human T-cell epitopes sequentially linked;
[3] the DNA according to [2], wherein the T-cell epitope peptide comprises seven human T-cell epitopes sequentially linked;
[4] the DNA according to any one of [1] to [3], wherein the storage protein promoter is selected from the group consisting of the glutelin GluB-1 promoter, the glutelin GluB-4 promoter, the 10 kD prolamin promoter, and the 16 kD prolamin promoter;
[5] the DNA according to any one of [1] to [4], wherein the ER-retention signal sequence is the KDEL sequence, the SEKDEL sequence, or the HDEL sequence;
[6] a vector for producing a plant comprising a T-cell epitope accumulated therein, wherein the vector comprises the DNA according to any one of [1] to [5];
[7] a host cell harboring the DNA according to any one of [1] to [5] or the vector according to [6];
[8] a method for accumulating an allergen-specific T-cell epitope in a plant, wherein the method comprises the step of introducing the DNA according to any one of [1] to [5] or the vector according to [6] into a plant;
[9] a method for accumulating a T-cell epitope in a plant, wherein the method comprises the following steps (a) to (c):
   (a) obtaining a DNA encoding an allergen-specific T-cell epitope peptide,
   (b) adding a DNA encoding a storage protein signal sequence to the 5′-end of the DNA obtained in (a) and/or a DNA encoding an ER-retention signal sequence to the 3′-end thereof, and
   (c) expressing the DNA of (b) under the control of a storage protein promoter in a plant;
[10] a method for accumulating a T-cell epitope in a plant, wherein the method comprises the following steps (a) and (b):
   (a) obtaing a DNA encoding an allergen-specific T-cell epitope peptide, and
   (b) inserting the DNA of (a) into a DNA region encoding a variable region of a plant storage protein and expressing it;
[11] the method according to any one of [8] to [10], wherein the storage protein is glutelin;
[12] the method according to any one of [8] to [11], wherein the epitope peptide comprises at least two, preferably seven, sequentially linked human T-cell epitopes;
[13] the method according to any one of [8] to [12], wherein the allergen is a Japanese cedar pollen allergen;
[14] the method according to [13], Wherein the Japanese cedar pollen allergen is Cry j1 and Cry j2;
[15] the method according to any one of [8] to [14], wherein the T-cell epitope is accumulated in an edible part of a plant;
[16] the method according to [15], wherein the edible part is a seed;
[17] the method according to any one of [8] to [16], wherein the plant is an angiosperm;
[18] the method according to [17], wherein the angiosperm is a poaceous plant;
[19] the method according to [18], wherein the poaceous plant is rice;
[20] a transgenic plant comprising a T-cell epitope accumulated therein, wherein the plant is produced by the method according to any one of [8] to [19];
[21] a transgenic plant which is a progeny or a clone of the plant according to [20];
[22] a cell derived from the plant according to [20] or [21];
[23] a breeding material of the plant according to [20] or [21];
[24] a seed of the plant according to [20] or [21];
[25] the seed according to [24], wherein the seed is thermostable;
[26] a rice comprising a T-cell epitope accumulated therein, wherein the rice is produced by the method according to [19], more preferably a rice comprising a T-cell epitope accumulated in albumen;
[27] a food composition for treating or preventing an allergic disease, wherein the food composition comprises the seed according to [24] or [25], or the rice according to [26], as an effective ingredient;
[28] the food composition according to [27], wherein the allergic disease is a type I allergy;
[29] the food composition according to [28], wherein the type I allergy is pollinosis or mite allergy;
[30] the food composition according to [29], wherein the pollinosis is associated with pollens of Japanese cedar, Japanese cypress, alder, ragweed, or cocksfoot;
[31] the food composition according to [27], wherein the allergy is a food allergy;
[32] the food composition according to [31], wherein the food is buckwheat;
[33] a method for producing a transgenic plant comprising a T-cell epitope accumulated therein using the method according to any one of [8] to [19];
[34] a method for producing a rice comprising a T-cell epitope accumulated therein using the method according to [19];
[35] a rice comprising an allergen-specific T-cell epitope accumulated in albumen;
[36] a food/drink product comprising the rice according to [35], wherein the product has an activity associated with the prevention, treatment, or alleviation of allergic diseases;
[37] the rice according to [35], wherein the allergen is a pollen allergen;
[38] a food/drink product comprising the rice according to [37], wherein the product has an activity associated with the prevention, treatment, or alleviation of allergic diseases; and
[39] the food/drink product according to [36] or [38], wherein the product is provided with an indication that it is to be used for preventing, treating, or alleviating allergic diseases such as pollinosis.

The present invention provides a method for accumulating an allergen-specific T-cell epitope in a plant.

This invention provides a method for expressing in a plant a T-cell antigen determinant (epitope) presented by antigen-presenting cells (macrophages) with an allergen as an antigen. As used in the context of the present invention, the phrase “allergen-specific T-cell epitope” refers to an allergen-derived T-cell epitope, more specifically to a T-cell epitope (peptide) presented by antigen-presenting cells with the allergen as an antigen.

In general, the term “allergen” refers to an antigenic substance causing allergic disease (allergic reaction). Allergens suitable for use in the present invention are not particularly limited and include not only naturally-occurring substances, such as proteins and glycoproteins, but also synthetic proteins. Examples of allergens found in nature are pollen (pollens of Japanese cedar, Japanese cypress, alder, ragweed, poaceous cocksfoot, etc.) allergens, animal (dog, cat, mouse, rat, horse, cattle, etc.)-derived allergens, insect allergens, parasite allergens, food allergens, fungal allergens, etc.

Preferred allergens for use in the present invention include pollen allergens, mite allergens, and food allergens, more preferably pollen allergens derived from Japanese cedar pollen. More specifically, the above-described Japanese cedar pollen allergens can be exemplified by Cry j1 (H. Yasueda, Y. Yui, T. Shimizu, T. Shida. Isolation and partial characterization of the major allergen from Japanese cedar (Cryptomeria japonica) pollen. J. Allergy Clin. Immunol. 1983; vol. 71, p. 77-86; T. Sone, N. Komiyama, K. Shimizu, T. Kusakabe, K. Morikubo and K. Kino Cloning and sequencing of cDNA coding for Cry jI, a major allergen of Japanese cedar pollen Biochem. Biophy. Res. Comm. 199, 619-625 (1994)) or Cry j2 (M. Sakaguchi, S. Inoue, M. Taniai, S. Ando, M. Usui, T. Matuhasi. Identification of the second major allergen of Japanese cedar pollen. Allergy 1990; vol. 45, p. 309-312; N. Komiyama, T. Sone, K. Shimizu, K. Morikubo and K. Kino, cDNA cloning and expression of Cry jII, the second major allergen of Japanese cedar pollen. Biochem. Biophy. Res. Comm. 201, 1021-1028(1994)). Further, examples of the aforementioned “foods” include buckwheat, wheat, egg, milk, and peanut.

A T-cell antigen determinant (occasionally referred to herein as an “epitope” or “epitope peptide”) used in the above-described method is usually an antigenic peptide, or a part of an antigenic peptide, which is presented on the cell surface of antigen-presenting cells after the allergen is degraded (antigen-processed) by the antigen-presenting cells. That is, epitopes of the present invention are not particularly limited in their amino acid sequences, so long as they are peptides recognized by T-cell receptors as allergen-derived antigenic peptides.

Although the T-cell epitopes of the present invention are not particularly limited, they are preferably human T-cell epitopes.

Epitopes (epitope peptides) of the present invention differ in their peptide lengths depending on allergen types and the like, so it is difficult to specify their lengths. In general, they comprise about 10 to 25, more preferably 12 to 19 amino acid residues.

Further, epitopes of the present invention are preferably those in which two or more human T-cell epitopes are sequentially linked. By linking two or more epitopes, the effect thereof on the immune-tolerance induction is expected to increase. More specifically, as shown in Examples described below, a peptide comprising seven epitopes linked (such as 7 Crp (SEQ ID NO: 1)) is preferably used as an epitope of the present invention. In addition, peptides made by linking an epitope peptide comprising multiple epitopes linked, such as 7 Crp, in tandem plural times, such as twice (SEQ ID NO: 2) or three times, can also be used in the present invention.

In the case of peptide immunization using epitopes, because different epitopes are by different individuals, depending upon their genotype, in the present invention a plurality of epitopes are preferably used so as to be effective in as many people as possible.

In a preferred embodiment of the method of the present invention, the epitope of the present invention is expressed in a plant body under the direction (control) of a storage protein promoter. More specifically, firstly, DNA encoding an allergen-specific human T-cell epitope peptide is obtained (process (a)), and secondly, DNA encoding a storage protein signal sequence is added to the 5′-end and/or DNA encoding ER-retention signal sequence to the 3′-end of the DNA obtained in the process (a) (process (b)). Subsequently, DNA obtained in process (b) is expressed under the control of a storage protein promoter in a plant (process (c)).

Herein, the term “storage protein” generally refers to a protein stored mainly as an energy source in the seeds of a plant. Examples of storage proteins are simple proteins, such as glutelin and prolamin. In the context of the present invention, a preferred storage protein is glutelin. The GenBank accession number of the gene encoding glutelin is X54314 (O. sativa GluB-1 gene for glutelin) and that for cDNA of the gene is XO5664.

In the present invention, promoters known in the art can be appropriately selected or modified to be used by one skilled in the art according to the types of genes desired to be expressed and the types of cells to be transduced. In a preferred embodiment of the present invention, a storage protein promoter can be used. One example of a storage protein promoter suitable for use in the context of the present invention is the glutelin GluB-1 promoter. This promoter is usually not less than 1.3 kb in length preferably not less than 2.3 kb, but is not particularly limited to this length so long as it is functionally equivalent to the promoter of the present invention. Preferably, the promoter is a 2.3 k GluB-1 promoter. Usually, the longer the promoter is, the higher the expression/accumulation efficiency of a protein encoded by the gene downstream of the promoter becomes. Because of its powerful promoter activity, the glutelin GluB-1 promoter can be preferably used for rice and other grains.

Preferred promoters useful in the methods of the present invention include, besides the above-described glutelin GluB-1, for example, the glutelin GluB-4 promoter, the 10 kD prolamin promoter, and the 16 kD prolamin promoter. As shown in the Examples described below, these promoters can be preferably used in the methods of the present invention. Information on nucleotide sequences for the promoters can be acquired as needed from patent or scientific references, or from public databases, such as GenBank (Japanese Patent Application No. 2003-373815; Plant Biotechnology Journal 2, 113-125 (2004) L. Q. Qu and F. Takaiwa, Evaluation of tissue specificity and expression strength of rice seed component gene promoters in transgenic rice; Glub-4 (Accession No.: AY427571, SEQ ID NO: 8); 10 k prolamin (Accession No.: AY427572, SEQ ID NO: 9); 16 kD prolamin (Accession No.: AY427574, SEQ ID NO: 10)).

Examples of DNA encoding an allergen-specific human T-cell epitope peptide suitable for use in the above-described process (a) include genomic DNA, cDNA, and chemically synthesized DNA. Genomic DNA and cDNA from living organisms, sources of allergens (e.g., rice), can be prepared by one skilled in the art using conventional methods. Genomic DNA can be prepared, for example, by extracting genomic DNA from a living organism as a source of allergen, preparing a genomic library (e.g., plasmid, phage, cosmid, BAC, PAC, or the like can be used as the vector), and developing it to perform colony hybridization or plaque hybridization using a probe prepared based on information on the nucleotide sequence of DNA encoding the allergen-specific human T-cell epitope peptide. Genomic DNA can also be prepared by performing PCR using specific primers for DNA encoding the allergen-specific human T-cell epitope peptide of the present invention. Further, cDNA can be prepared, for example, by synthesizing cDNA based on mRNA extracted from a living organism as a source of allergen, inserting this cDNA into a vector, such as λZAP, to make cDNA library, and developing it to perform colony hybridization or plaque hybridization, or by PCR, as described above.

It is also possible to appropriately synthesize DNA encoding the epitope peptide of the present invention artificially, preferably based on information on the amino acid sequence of the peptide. In this case, the objective DNA can be synthesized with reference to codons frequently used in storage protein genes, taking the degeneracy of amino acids and such into consideration. Further, when an epitope peptide of the present invention, e.g., 7 Crp, is used, a DNA sequence encoding the peptide can be prepared using codons used in the rice seed storage protein gene in high frequency so as to be efficiently translated in rice seeds.

Codons preferably used in the present invention include, but not particularly limited to, for example, the codons in Table 1.

TABLE 1

| | | | | |
|---|---|---|---|---|
| Ala(A) | Asp(D) | Arg(R) | Asn(N) | Cys(C) |

| | | | | |
|---|---|---|---|---|
| GCA | GAT | CGT | AAT | TGC |
| GCT | | AGG | AAC | TGT |
| | | AGA | | |

| | | | | |
|---|---|---|---|---|
| Gly(G) | Glu(E) | Gln(Q) | Leu(L) | Stop codon |

| | | | | |
|---|---|---|---|---|
| GGC | GAA | CAA | CTC | TAA |
| GGA | GAG | CAG | CTT | TGA |
| | | | CTA | TAG |
| | | | TTG | |

| | | | | |
|---|---|---|---|---|
| His(H) | Ile(I) | Phe(F) | Pro(P) | Thr(T) |

| | | | | |
|---|---|---|---|---|
| CAT | ATC | TTC | CCA | ACT |
| CAC | ATT | | CCC | ACA |
| | | | CCT | ACC |

| | | | | |
|---|---|---|---|---|
| Lys(K) | Ser(S) | Trp(W) | Tyr(Y) | Val(V) |

| | | | | |
|---|---|---|---|---|
| AAG | AGT | TGG | TAC | GTT |
| | TCT | | TAT | GTA |
| | AGC | | | |

Met(M)
ATG

One skilled in the art can appropriately perform DNA synthesis using a commercial DNA synthesizer or the like.

Regarding the storage protein signal (peptide) sequence in the above-described process (b), various known storage protein signal sequences can be appropriately used. Information on amino acid sequences of storage protein signal sequences of the present invention can be readily obtained by one skilled in the art from known published references and the like. Storage protein signals function to locate the epitope peptide of the present invention to endoplasmic reticulum, thereby preventing the peptide from being located to cytoplasm and degraded or secreted to the outside of the cell.

Regarding the storage protein signal sequence of the present invention, the signal sequence of glutelin (GluB-1) protein can be preferably used. Specifically, storage protein signal sequences usable in the present invention include the following sequence: MASSVFSRFSIYFCVLLLCHGSMA (SEQ ID NO: 3).

It is also possible to use the signal sequence of another glutelin (GluA-2): MASINRPIVFFTVCLFLLCDGSLA (SEQ ID NO: 4) or that of the 26 kD globulin: MASKVVFFAAALMAAMVAISGAQ (SEQ ID NO: 5).

Further, regarding the ER-retention signal sequence of the present invention, for example, the KDEL sequence, the SEKDEL sequence, or the HDEL sequence can be used; however, the present invention is not particularly limited to these examples. DNA encoding the ER-retention signal sequence of the present invention may contain, for example, a 3′-noncoding region downstream of a DNA encoding the KDEL sequence. Although this 3′-nonconding region is not particularly limited, it is usually in the range of about 100 to 1000 bp long. As an example, a DNA encoding an ER-retention signal sequence of the present invention is a DNA comprising a DNA encoding the KDEL sequence and the glutelin 3′-noncoding region of about 650 bp in length downstream of that DNA. In general, as the above-described 3′-noncoding region, the 3′-noncoding regions of genes of storage proteins, such as glutelin, can be preferably used. The NOS terminator or the 35S CaMV terminator can also be used. The aforementioned sequences function to improve the accumulation amount of foreign proteins in storage parts, such as seeds.

DNA encoding the above-described storage protein signal sequence or ER-retention signal sequence can be obtained (synthesized) by one skilled in the art by appropriately using a commercial DNA synthesizer or the like, taking the degeneracy of amino acids and such into consideration.

Further, the addition of DNA encoding a storage protein signal sequence to the 5′-end, and DNA encoding an ER-retention signal sequence to the 3′-end of DNA obtained in the process (a) can be performed by one skilled in the art using known genetic engineering techniques. The aforementioned “5′-end” or “3′-end” is usually defined as the end to indicate that the direction receiving the tanscription control from the promoter is 5′-end ->3′-end. Accordingly, the DNA end on the promoter side (direction) is defined as the “5′-end.”

In the process (c), expression of DNA under the control of a storage protein promoter in a plant body can generally be performed by introducing DNA in which the storage protein promoter is linked thereto, so as to enable the expression of the DNA into the plant body. DNA desired to be expressed can readily be located downstream of the promoter so as to receive the promoter control, by one skilled in the art using general genetic engineering techniques.

Although plants suitable for use in the context of the methods of the present invention are not limited to any particular species, they are typically angiosperms, preferably monocotyledons, more preferably poaceous plants. Plants of the present invention may be dicotyledons; for example, it is possible to accumulate the epitope in plants such as legumes by using a seed promoter of a dicotyledon (for example, cotyledon- or embryo-specific). Further, although poaceous plants can be exemplified by grains, such as rice, wheat, barley, and corn, the more preferred plant for use in the present invention is rice. In the method of the present invention, it is possible to accumulate the epitope in a great variety of plants by suitably selecting the promoter to be used, taking the various types of plants into consideration.

In the method of the present invention, when the part in plant body in which the epitope is accumulated is an edible part, it is possible for humans to absorb the epitope of the present invention easily into the body, for example, by eating that part. For example, when the plant of the present invention is rice, it is possible to accumulate the epitope in albumen.

The method of the present invention is useful for preferentially accumulating a T-cell epitope in the edible part of a plant body. Although edible parts differ according to the types of plants and are not particularly limited, they include seeds, leaves, and roots.

More specifically, examples of suitable accumulation regions include albumens for rice, wheat, corn, and such; cotyledons and embryos for beans such as soybean; tubers of potatoes and such; carrot roots; and fruits such as tomatoes and bananas.

DNA capable of expressing in a plant body the epitope of the present invention used in the methods of the invention is also included in the present invention. Such DNA can be exemplified by DNA having a structure in which any one of the following DNA is placed under the control of a storage protein promoter.
(a) DNA in which DNA encoding the storage protein signal sequence is added to the 5′-end of DNA encoding the allergen-specific T-cell epitope peptide, and/or DNA encoding the ER-retention signal sequence to the 3′-end thereof;
(b) DNA encoding a polypeptide in which the storage protein signal sequence is added to the N-terminal of the allergen-specific T-cell epitope peptide, and/or the ER-retention signal sequence to the C-terminal thereof.

The phrase “DNA is placed under-the control of a storage protein promoter” indicates that the storage protein promoter and the DNA are linked so as to enable the expression of the DNA. That is, it means that the promoter and the DNA downstream thereof are linked such that the expression of the DNA downstream of the promoter is induced when the promoter is transcriptionally activated.

Further, in another embodiment of the present invention, there is provided a method of inserting a T-cell epitope into a storage protein and expressing/accumulating the epitope as a part of the storage protein.

In a preferred embodiment of this method, firstly DNA encoding the allergen-specific T-cell epitope peptide is obtained, and secondly the DNA is inserted into a DNA region encoding a variable region of a plant storage protein to be expressed.

In this method, the insertion site for the epitope in a storage protein is preferably a variable region of the storage protein. The phrase “variable region” refers to a region that has a great variety in terms of the types and lengths of amino acid sequence acquired over the course of evolution. Therefore, since the insertion of foreign peptides does not affect the three-dimensional structure, it is possible to accumulate a foreign peptide as a part of the storage protein. In addition, since the foreign peptide behaves as a part of the storage protein, it is possible to accumulate it in the same storage site as that of the peptide-introduced storage protein.

Variable regions used in the present invention include, for example, in the case of rice glutelin, three regions of the acidic subunit (in the case of the glutelin GluB-1, regions of amino acids 140, 210, and 270 to 310 as counted from the N-terminal) and the C-terminal region of the basic subunit. Accordingly, a foreign peptide to be expressed can be inserted into these regions. It is also possible to insert the epitope of the present invention, one each into the above-described respective variable regions. Glutelin belongs to the 11S globulin family (soybean glycinin and oats globulin are members), and, thus, a foreign peptide can be inserted into the variable regions exemplified above of other proteins belonging to this family. However, in the case of rice globulin (as distinguished from oats globulin), it is possible to insert the epitope into the variable region located about 110 amino acids from the N-terminal.

As one example of the above-described method, a human T-cell epitope is accumulated as a part of the glutelin storage protein by inserting one 7 Crp coding region of a 96-amino acid sequence (1×7 Crp) or two thereof in tandem (2×7 Crp) into the region encoding the amino acid residues Nos. 275 to 305 (the acidic subunit coding region) of the glutelin precursor coding region of the pREE99 cDNA clone of the glutelin GluA-2 gene, and expressing it as a part of glutelin.

In this method, insertion of DNA encoding the epitope of the present invention into a DNA region encoding the variable region of the storage protein can be readily performed by one skilled in the art using standard genetic engineering techniques.

DNA encoding a polypeptide having a structure in which the T-cell epitope used in the above-described method is inserted into the storage protein is also included in the present invention. Such DNAs include, for example, (c) DNA encoding a polypeptide having a structure in which an allergen-specific T-cell epitope peptide is inserted into the amino acid sequence (preferably the variable region) of the storage protein under the control of the storage protein promoter. Usually, the promoter originally present upstream of the storage protein gene can be used as the promoter.

The present invention also provides a vector comprising DNA of the present invention and a host cell harboring a DNA or vector of the present invention. Vectors of the present invention are not particularly limited, so long as they are capable of stably retain DNA of the invention. Vectors of the present invention can be prepared by one skilled in the art by cloning the above-described DNAs into known various vectors, appropriately considering types of plants in which the DNAs are desired to be expressed. Insertion of DNA of the present invention into known vectors can be performed by a standard method, for example, by ligase reaction using a restriction enzyme site (Current protocols in Molecular Biology edit. Ausubel et al. (1987) Publish John Wiley & Sons. Section 11.4-11.11).

Examples of vectors of the present invention include the vector pGluBsig7CrpKDEL described in the Examples below. The vector of the present invention can be used in the epitope accumulation method of the present invention, and is, therefore, useful as a vector for preparing T-cell epitope accumulated plants.

Host cells into which the vector of the present invention is to be introduced are not particularly limited, and various known cells may be used according to objectives. The phrase “host cells” as used herein includes various forms of plant cells, including plant cells of all forms capable of regenerating a plant body. Host cells include, for example, suspended culture cells protoplasts, shoot primordia, multiple shoots, hairy roots, and calluses, but are not limited to them. When the preservation, reproduction, and the like of the vector of the present invention are intended, host cells of the present invention need not necessarily be plant-derived cells, and may be, for example, E. coli, yeast, or animal cells.

Introduction of vectors into host cells can be performed by known methods, such as the calcium phosphate precipitation method, the electroporation method (Current protocols in Molecular Biology edit. Ausubel et al. (1987) Publish. John Wiley & Sons. Section 9.1-9.9), lipofectamine method (GIBCO-BRL), and the microinjection method.

The present invention also relates to a method for accumulating a T-cell epitope in a plant comprising the step of introducing a DNA or vector of the present invention into a plant, as well as a method for producing a plant comprising a T-cell epitope accumulated therein comprising the step of introducing a DNA or vector of the present invention into a plant. In a preferred embodiment of the present invention, there will be provided a method for producing a transgenic plant comprising a T-cell epitope accumulated therein and a method for producing rice comprising a T-cell epitope accumulated therein using techniques of the present invention.

When producing a transformant plant body comprising an allergen-specific human T-cell epitope accumulated therein using DNA of the present invention, for example, DNA of the present invention may be inserted into an appropriate vector, which may then be introduced into a plant cell, and the transformant plant cell thus obtained may be cultivated (regenerated). Cultivation (regeneration) of a plant body can be carried out by a method known to one skilled in the art according to the types of plant cells (see Toki et al. (1995) Plant Physiol. 100: 1503-1507). For example, as for rice, several techniques of producing transformant plant body have been established, such as the method of introducing a gene into protoplast with polyethylene glycol to grow (regenerate) a plant body (Datta, S. K. (1995) In Gene Transfer To Plants (Potrykus, I. and Spangenberg Eds.) pp. 66-74); the method of introducing a gene into protoplast by electric pulse to grow (regenerate) a plant body (Toki et al. (1992) Plant Physiol. 100, 1503-1507); the method of directly introducing a gene into cells by particle-gun method to grow (regenerate) a plant body (Christou et al. (1991) Bio/technology, 9: 957-962); and the method of introducing a gene via Agrobacterium to grow (regenerate) a plant body (Hiei et al. (1994) Plant J. 6: 271-282). These techniques are broadly used in the technical field of the present invention. In the present invention, these methods can be appropriately used. When the Agrobacterium method is used, for example, the method of Nagel et al. is preferably employed (Microbiol. Lett., 1990, 67, 325). According to this method, a vector is introduced into Agrobacterium cells, and subsequently the transformed Agrobacterium cells are introduced into plant cells by known methods, such as the leaf disc method.

Further, plants to be transduced with the DNA or vectors of the present invention may be explants. Cultured cells may be prepared from these plants, and then, the DNA or vector can be introduced into the cultured cells. Examples of “plant cells” of the present invention include plant cells of leaf, root, stem, flower, and scutellum in the seed, callus, suspended culture cells, etc.

Further, in order to efficiently select plant cells transformed by introduction of a DNA or nucleic acid of the present invention, a DNA or vector of the present invention preferably contains an appropriate selective marker gene, or is introduced into plant cells together with a plasmid vector containing such a selective marker gene. Examples of selective marker genes used for this purpose are the hygromycin phosphotransferase gene resistant to the antibiotic hygromycin; the neomycin phosphotransferase gene resistant to kanamycin or gentamycin; the acetyl transferase gene resistant to the herbicide phosphinothricin, and the like.

Plant cells transduced with a recombinant vector may be plated and cultured on a known selective medium containing an appropriate selective drug according to the type of the selective marker gene used. Through this method, transformed plant cells in culture can be obtained.

A plant body regenerated from the transformant cells is then cultured in the conditioned medium. Subsequently, the regenerated plant body thus acclimatized is cultivated under normal cultivation conditions to obtain the plant body. It is possible for this plant body to ripen, bear fruits, and yield seeds.

The presence of DNA of the present invention introduced into the transformant plant body thus cultivated (regenerated) can be confirmed by the known PCR method and Southern hybridization method. In this case, extraction of DNA from the transformant plant body can be performed according to the known method of J. Sambrook et al. (Molecular Cloning, 2^{nd} ed., Cold Spring Harbor Laboratory Press, 1989).

Further, the present invention provides transgenic plants (transformant plants) produced by the method of the present invention, in which a T-cell epitope is accumulated, and cells derived from such plants. For example, a plant comprising a Japanese cedar pollen allergen T-cell epitope accumulated in seeds by the method of the present invention is useful as a Japanese cedar pollinosis lenitive crop.

Once a transformant plant body having a T-cell epitope accumulated therein in accordance with the present invention is obtained, it is possible to produce progenies from the plant body by sexual or asexual reproduction. It is also possible to obtain breeding materials (such as seeds, fruits, cuttings, tubers, tuberous roots, stumps, calluses, and protoplasts) from the plant body and progeny or clone thereof, and mass-produce the plant body based on these materials. The present invention includes progenies or clones of the transgenic plant in which a T-cell epitope produced by the method of the present invention is accumulated, cells, breeding materials as well as seeds derived from the transgenic plant or progenies and clones thereof. Seeds of the present invention are expected to have thermostability. A preferred embodiment of the present invention includes, for example, rice (rice plant) in which an allergen-specific human T-cell epitope is accumulated in albumen.

Further, the present invention provides food compositions and food/drink products having the prophylactic, curing, or lenitive action against allergic diseases. Food compositions or food/drink products of the present invention are composed of the parts (e.g., seed, rice) of the plant in which the epitope produced by the method of the present invention is accumulated or extracts containing the epitope extracted from these parts or processed products thereof. More specifically, they are food compositions or food/drink products for the treatment or prevention of allergic diseases containing seed or rice accumulated with the epitope obtained by the method of the present invention or ingredients extracted from them. The term “composition” in the context of the present invention is not necessarily further supplemented with multiple types of substances besides seed or rice, and may be a food composed of only the seed or rice of the present invention.

It is also possible to process the food compositions or food/drink products of the present invention by cookery, such as heating. They can also be processed to be used as a health-promoting food, functional food, specific health-preservation food, nutritional supplement food, and the like by mixing them with food-hygienically acceptable additives. The food composition is appropriately provided with additives, such as a stabilizer, preservative, colorant, food flavor, and/or vitamins, then mixed, and can be processed by a standard method into forms suitable for the composition, such as a tablet, particle, granule, powder, capsule, liquid, cream, and drink.

A preferred embodiment of the present invention is a food/drink product containing the rice accumulated with an allergen (such as Japanese cedar pollen allergen)-specific T-cell epitope having the prophylactic, curing, or lenitive action against allergic diseases (e.g., pollinosis). The food and drink include processed products made of the rice of the present invention as the material and can be exemplified by rice cakes (dumpling, sliced rice cake, polished rice flour, glutinous rice flour), rice crackers, rice noodles, refined sake, brown rice tea, rice bran, noodles (udon), etc.

Food/drink products of the present invention are preferably include an indication that they are intended to be used for preventing, treating, or mitigating allergic diseases (e.g., pollinosis).

Pharmaceutical compositions for the treatment or prevention of allergic diseases containing the parts (such as seed and rice) accumulated with the plant-derived epitope produced by the method of the present invention as an effective ingredient are also included in the present invention.

In the present invention, allergic disease is a general term for diseases associated with allergic reactions. Examples of allergic disease include pollinosis, bronchial asthma, food allergy, allergic rhinitis, and insect allergy. In addition, allergic diseases can be classified into diseases showing type I to IV allergic reactions. Allergic diseases of the present invention are not particularly limited, but are preferably type I allergy. Examples of type I allergy are pollinosis, mite-allergy, bronchial asthma, food allergy, and allergic rhinitis. In the present invention, preferred allergic diseases include Japanese cedar pollinosis and mite allergy. The methods of the present invention using the allergen-specific epitope corresponding to the above-described diseases are expected to be applied to the treatment what is called tailor-made therapy.

### BRIEF DESCRIPTION OF THE DRAWINGS

FIG. 1 represents a set of drawings and a photograph showing the analytical results of 7 Crp expression in the transformants produced by the vector pGluBsig7CrpKDEL of the present invention. The upper panel schematically shows the structure of the plasmid pGluBsig7CrpKDEL. 7 Crp is expressed under the control of the 2.3 k promoter for the glutelin GluB-1 gene. The middle panel is a graph showing the quantitation results of 7 Crp accumulation amount in fully ripened seeds of the transformants obtained, relatively high accumulation amounts being observed in seeds of the lines #1, #10, #15, #17, #31, #34, etc. The lower panel is a photograph showing the results of northern analysis of transcripts of the 7 Crp gene in seeds at the grain-filling stage (about 15 days after flowering) of the transformant of T0 generation.

FIG. 2 represents a set of drawings and a photograph showing the analytical results of 7 Crp expression in the transformants produced by the vector pGluBsig7Crp of the present invention. The upper panel schematically shows the structure of the plasmid pGluBsig7Crp, in which the KDEL sequence is not added to the 3′-end of the 7 Crp gene. The middle panel is a graph showing the quantitation results of 7 Crp accumulation amount in fully ripened seeds of the transformants obtained. Compared to the case in FIG. 1 in which the KDEL sequence is added to the 3′-end of the 7 Crp gene, the accumulation amount of 7 Crp is decreased. The lower panel is a photograph showing the results of northern analysis of transcripts of the 7 Crp gene in seeds at the grain-filling stage of the transformant rice of T0 generation.

FIG. 3 represents a photograph showing the results of Southern analysis of the genomic DNA of the transformants produced by the vector pGluBsig7CrpKDEL of the present invention. Genomic DNAs (10 μg each) extracted from leaves of the non-transformant rice and transformant lines #1, #10, #17, and #34 were digested with the restriction enzyme Sac I, and, after fractionated by 0.9% (w/v) agarose electrophoresis, blotted onto a nylon membrane. Subsequently, the 7 Crp gene was detected with the radiolabelled full-length DNA of the 7 Crp gene as a probe.

FIG. 4 is a set of photographs showing the expression patterns of glutelin and the 7 Crp peptide at the grain-filling stage of seeds by western analysis. The total protein of seeds of the transformant #10 produced by the vector pGluBsig7CrpKDEL of the present invention was extracted 5, 10, 15, 20, and 25 days after flowering, fractionated by SDS-PAGE, and blotted onto a PVDF membrane. Subsequently, glutelin or 7 Crp peptide was detected using anti-glutelin antibody or anti-7 Crp antibody.

FIG. 5 represents a set of photographs showing the expression patterns of the glutelin gene and the 7 Crp gene at the grain-filling stage of seeds by northern analysis. Respective total RNAs of the seed, leaf, and stem of the transformant #10 produced by the vector pGluBsig7CrpKDEL of the present invention were extracted, fractionated by agarose-gel electrophoresis, and blotted onto a nylon membrane. Subsequently, the transcript of the glutelin gene or the 7 Crp gene was detected using the radiolabelled full-length DNA of the glutelin or 7 Crp gene as a probe. In the upper photograph, 25S and 17S rRNAs were visualized, while, in the middle photograph, the analytical result of transcript of the glutelin gene, and, in the lower photograph, that of the 7 Crp gene are shown, respectively.

FIG. 6 represents a set of photographs showing the analytical results of accumulation sites of the 7 Crp peptide in seeds of the transformant #10 produced by the vector pGluBsig7CrpKDEL of the present invention. The left photograph shows results of western analysis for the protein fractions extracted from albumen, embryo, glume, and leaf, respectively, using an anti-7 Crp antibody. In the right photographs, the results of tissue immunostaining of the seed section at the grain-filling stage using the anti-7 Crp antibody are shown. In order to visualize the 7 Crp signal, the anti-rabbit IgG alkaline phosphatase-labelled antibody was used.

FIG. 7 is a photograph showing the results comparing the 7 Crp peptide accumulation amounts in the seeds of T1, T2, and T3 generations of the transformants produced by the vector pGluBsig7CrpKDEL of the present invention. As to the transformants (lines #1-3, #10-1, #10-4, and #17-1) selected as homozygotes and cultivated, the total proteins were extracted from seeds of respective generations and subjected to western analysis using the anti-7 Crp antibody.

FIG. 8 represents a set of photographs showing the analytical results of the thermostability of the 7 Crp peptide accumulated in seeds of the transformant produced by the vector pGluBsig7CrpKDEL of the present invention. After the seeds of transformant line #10 were heated in boiling water for 20 min, the total protein was extracted, and fractionated by SDS-PAGE. As a control, the total protein was extracted from seeds without heat treatment. In the left photograph, the protein was visualized using CBB, while, in the right photograph, 7 Crp was detected by western analysis using the anti-7Crp antibody.

FIG. 9 represents a set of photographs showing analytical results of N-linked sugar chain for the 7 Crp peptide accumulated in seeds of the transformant produced by the vector pGluBsig7CrpKDEL of the present invention. First, the total protein fraction extracted from seeds of transformant line #10 was reacted with N-glycosidase F. As the control substrate for the glycosidase reaction, transferrin and ribonuclease B, N-linked glycoproteins, were used. Subsequently, changes in the substrate molecular weight were analyzed by SDS-PAGE before and after the reaction with the enzyme. In the left photograph, the 7 Crp peptide was visualized by western analysis using the anti-7 Crp antibody, and, in the right photograph, the control substrates were visualized by staining with CBB, respectively.

FIG. 10 represents a series of photographs showing analytical results of the effect of 7 Crp peptide expression on the rice allergenic protein expressions. After total proteins extracted from seeds of the nontransformant rice and transformant rice line #10 were fractionated by SDS-PAGE respectively, allergenic proteins were detected by western analysis using specific antibodies recognizing the respective rice allergenic proteins.

FIG. 11 represents a set of drawings and a photograph showing the analytical results of 7 Crp expression in the transformants produced by the vector pAGPasesig7CrpKDEL of the present invention. The upper panel schematically represents the structure of the plasmid pAGPasesig7CrpKDEL, in which 7 Crp expression is controlled by the promoter for ADP-glucose pyrophosphorylase. The middle panel is a graph showing the quantitation results of 7 Crp accumulation amount in the transformant fully ripen seeds obtained. Although, compared to the case using the 2.3 k GluB-1 promoter, the accumulation amount of 7 Crp was decreased, its accumulation was observed in many lines. The lower panel shows northern analytical results for transcript of the 7 Crp gene in seeds at the grain-filling stage of the transformants of T0 generation.

FIG. 12 represents a set of drawings and a photograph showing the analytical results of 7 Crp expression in the transformants produced by the vector pREE99 2×7 Crp of the present invention. The upper panel schematically represents the structure of the plasmid pREE99 2×7 Crp. Two 7 Crp are inserted in tandem into the variable region of cDNA clone pREE99 of the glutelin GluA-2 gene. The middle panel is a graph showing the quantitation results of 7 Crp accumulation amount in fully ripened seeds of the transformants obtained, in which two 7 Crp inserted into the variable region accumulate as a part of glutelin. The lower panel shows northern analytical results for transcripts of the 7 Crp gene inserted into pREE99 in seeds at the grain-filling stage of the transformants of T0 generation.

FIG. 13 represents a set of drawings and photographs concerning comparisons of accumulation amounts of T-cell epitope peptide due to the difference in types of promoters. The upper panel is a series of drawings showing the structures of genes using respective promoters. The lower panel is a series of gel electrophoresis photographs comparing accumulation amounts of T-cell epitope peptide.

FIG. 14 represents a set of drawings and photographs concerning comparisons of accumulation amounts of T-cell epitope peptide, due to the localization site thereof. The upper panel is a series of drawings showing the structures of respective genes, in which ChiChi represents “chitinase.” The lower panel is a series of photographs comparing accumulation amounts of T-cell epitope peptide due to the localization site thereof.

FIG. 15 represents a set of graphs showing the induction results of immune tolerance by oral administration of a recombinant rice expressing the T-cell epitope peptide 7 Crp. The left graph shows the results of measuring T-cell proliferative reaction, while the right graph shows those of measuring IgE level.

### BEST MODE FOR CARRYING OUT THE INVENTION

The present invention is illustrated in more detail below with reference to Examples, but should not be construed as being limited thereto.

### EXAMPLE 1

### Preparation of a T-cell Epitope-Linked Peptide Expression Plasmid and Its Introduction into Rice Kita-Ake

An expression plasmid for expressing a Japanese cedar pollen allergen T-cell epitope-linked peptide in rice seeds was prepared. After a promoter for the rice seed major protein glutelin GluB-1 (although 1.3 kb promoter had usually been used, the 2.3 kb promoter was used in the present invention; promoter activity being elevated 5-fold or more), a signal sequence, and a T-cell epitope-linked peptide gene were linked, the ER-retention signal KDEL sequence, which has the function to improve the accumulation amount of a foreign gene product in seeds, was added to the 3′-end of the T-cell epitope-linked peptide to produce the expression plasmid pGluBsig7CrpKDEL. The DNA nucleotide sequence used in Examples comprising the 2.3 kb GluB-1 promoter sequence, the glutelin signal sequence, the 7 Crp epitope sequence, the KDEL sequence, and the 0.6 k GluB-1 3′-sequence, is shown in SEQ ID NO: 6, and the amino acid sequence encoded by the DNA is shown in SEQ ID NO: 7.

In order to examine actions of the signal sequence and the KDEL sequence toward the expression of the T-cell epitope-linked peptide, the plasmids pGluB7CrpKDEL and pGluBsig7Crp lacking the signal sequence and the KDEL sequence in pGluBsig7CrpKDEL respectively were also constructed.

Further, a sequence for the 7 Crp peptide was inserted into a variable region of the acidic subunit of glutelin (GluA-2), the major storage protein of rice, so as to express the 7 Crp peptide as a part of glutelin. These plasmids were introduced into the rice Kita-ake by the Agrobacterium method and transformants were selected with the hygromycin-resistance as an indicator. In these analyses, transformants of 30 lines or more were used for the respective constructs.

### EXAMPLE 2

### Detection of a T-cell Epitope-Linked Peptide in Transformant Seeds

Total RNA fractions were recovered from seeds at the grain-filling stage of the pGluB7CrpKDEL transformants having no signal sequence, and northern analysis was performed using the T-cell epitope-linked peptide gene as a probe. As a result, transcripts were detected in 27 out of 32 lines, so that accumulation of a T-cell epitope-linked peptide in seeds was expected.

Therefore, after proteins were extracted from the fully ripened seeds and fractionated by electrophoresis, they were visualized by CBB staining or western blot analysis using a specific antibody. As a result, contrary to expectations, no T-cell epitope-linked peptide signal was detected. The reason for this is probably that, due to lack of the signal sequence, the T-cell epitope-linked peptide localizes not to the rough endoplasmic reticulum but to cytoplasm to be degraded, or, alternatively, is excreted to the outside of cells.

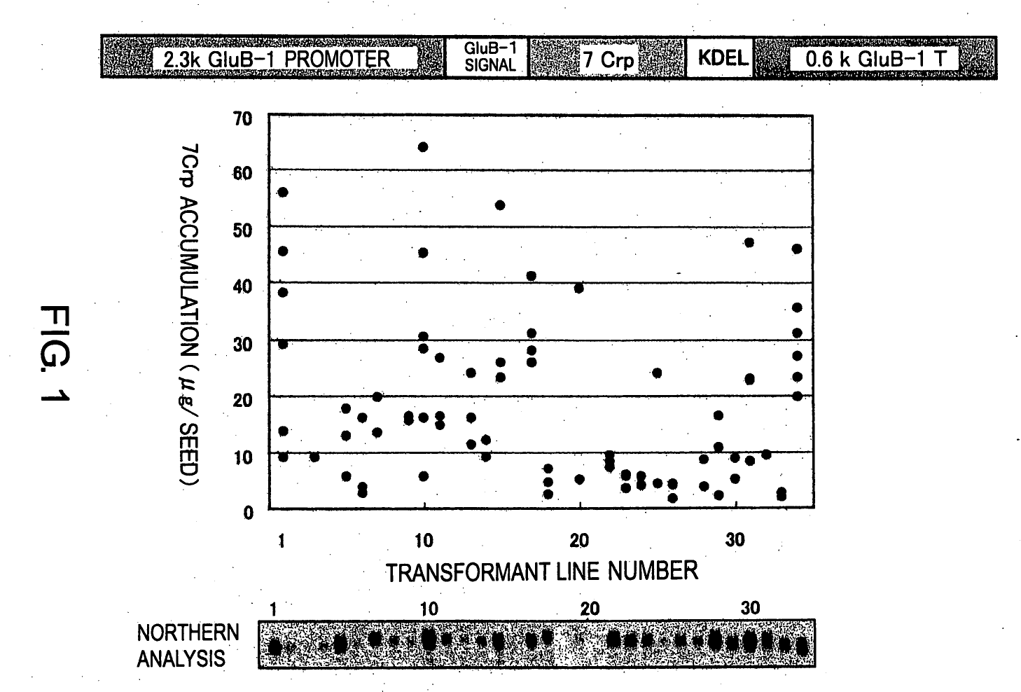

Next, the pGluBsig7CrpKDEL transformants having the signal sequence were analyzed. As a result of northern analysis for the total RNA fractions prepared from seeds at the grain-filling stage, transcript signals were detected in 29 out of 34 lines with strong signals being observed in lines #1, #5, and #10 in particular (FIG. 1). As a result of analyzing the total protein prepared from fully ripened seeds of line #10, a signal showing the mobility corresponding to the molecular weight of about 11,000, which was not observed with the nontransformant, was detected. Since this apparent molecular weight coincides well with the molecular weight 11,229 presumed from the gene sequence of the T-cell epitope-linked peptide, this signal was judged to be derived from the T-cell epitope-linked peptide.

Next, with the signal intensity in western blot analysis as an indicator, accumulation amount of T-cell epitope-linked peptide in seeds was estimated. As a standard protein for the signal intensity, a T-cell epitope-linked peptide-histidine tag fusion protein expressed in E. coli and purified was used. As a result, in lines #1, #10, #15, #17, #31, #34, and so on, relatively large amounts of T-cell epitope-linked peptide were accumulated. Among them, in seeds of line #10 in which the highest accumulation amount was detected, accumulation of 60 μg of T-cell epitope-linked peptide corresponding to 4% of the total seed protein was confirmed.

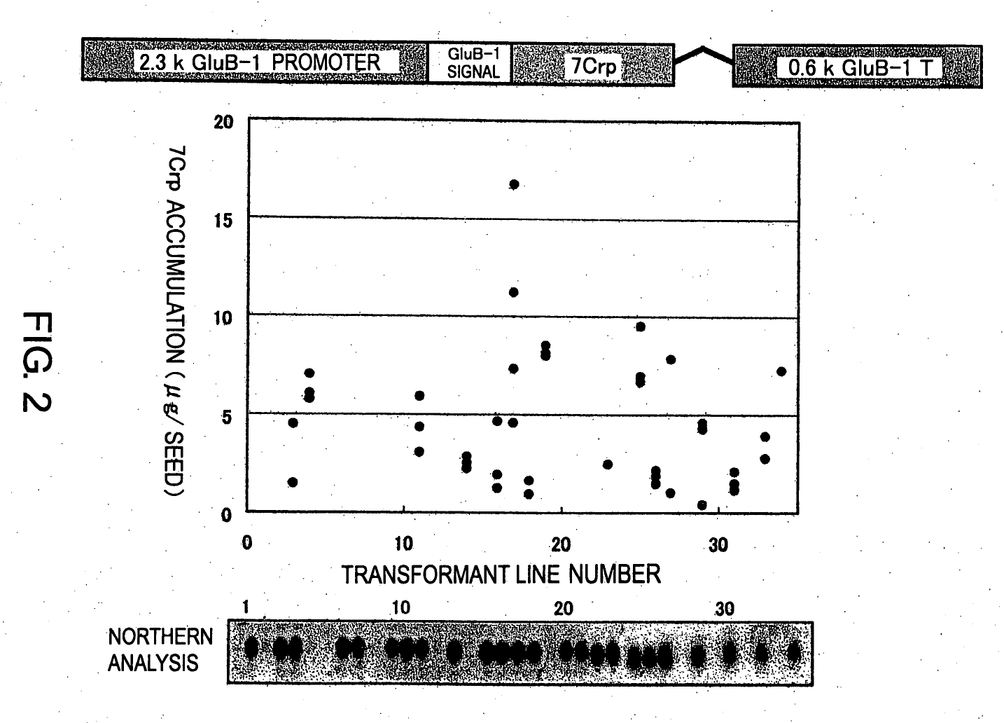

On the other hand, as a result of analysis for the pGluBsig7Crp transformants lacking the KDEL sequence, transcripts were detected in 25 out of 38 lines, and, as a result of western analysis for the fully ripened seed protein, accumulation of T-cell epitope-linked peptide was observed. However, as compared to the pGluBsig7CrpKDEL transformants having the KDEL sequence, accumulation amount of T-cell epitope-linked peptide was greatly decreased to 16 μg corresponding to 1.1% of the total seed protein even in seeds of the line showing the highest accumulation amount (FIG. 2).

From the results above, it was proved that a T-cell epitope-linked peptide was successfully produced in rice seeds by introduction of the plasmid pGluBsig7CrpKDEL, that the signal sequence is essential for the expression of a T-cell epitope-linked peptide, and that accumulation amount thereof is improved by the addition of the KDEL sequence.

### EXAMPLE 3

### Detection of a T-cell Epitope-Linked Peptide Gene Introduced into Rice

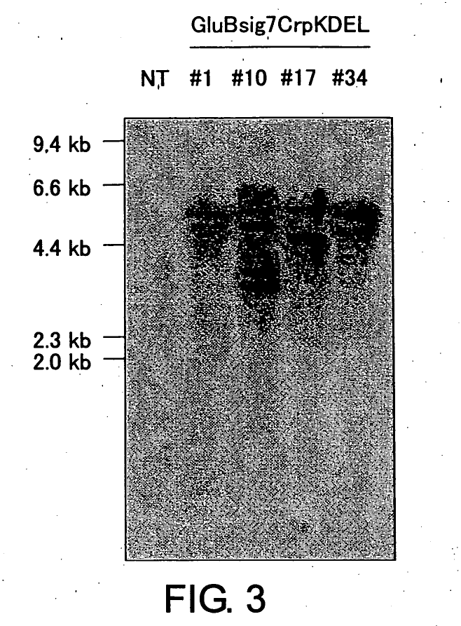

In order to detect a T-cell epitope-linked peptide gene introduced into the transformant genome and identify its copy number, analysis of the transformant genomic DNA was performed by Southern blot technique. Among transformants thus obtained, genomic DNA was prepared from leaves of transformant lines showing high level of T-cell epitope-linked peptide expression, and, after treatment with the restriction enzyme Sac I, Southern blot analysis was carried out using a whole region of the T-cell epitope-linked peptide gene as a probe. Since the plasmid used in the transformation is cleaved by Sac I only at one site, the number of bands detected by Southern analysis corresponds to the copy number of a T-cell epitope-linked peptide gene introduced into the rice genome. As a result of Southern blot analysis, among pGluBsig7CrpKDEL transformants, it was confirmed that in #1 two copies; in #10 four copies; and in #17 two copies of the T-cell epitope-linked peptide gene were introduced into the transformant genome. And, with the pGluBsig7Crp transformants, there were confirmed the introduction of two copies in #17, one copy in #19 and two copies of the gene in #25, respectively (FIG. 3).

### EXAMPLE 4

### Expression Characteristic of a T-cell Epitope-Linked Peptide in Rice Seeds

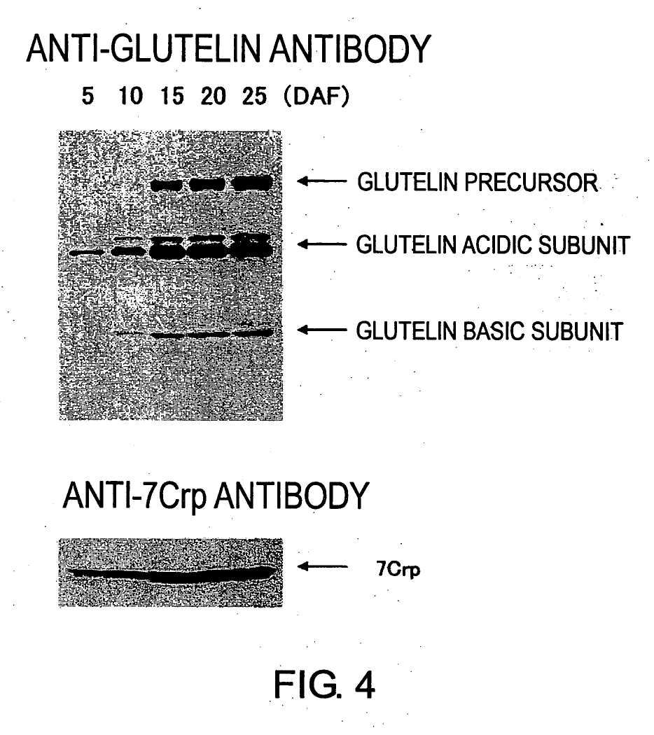

Using the pGluBsig7CrpKDEL transformant line #10 showing the highest expression amount of Tell epitope-linked peptide to analyze the expression progress thereof in the seed at the grain-filling stage, proteins were extracted from seeds at various time points after flowering and analyzed by western blot. T-cell epitope-linked peptide signal was detected for the first time 5 days after flowering and subsequently increased gradually, reaching the level of fully ripened seeds. This result coincides well with the expression progress of the GluB-1 protein analyzed using the specific antibody recognizing this protein (FIG. 4).

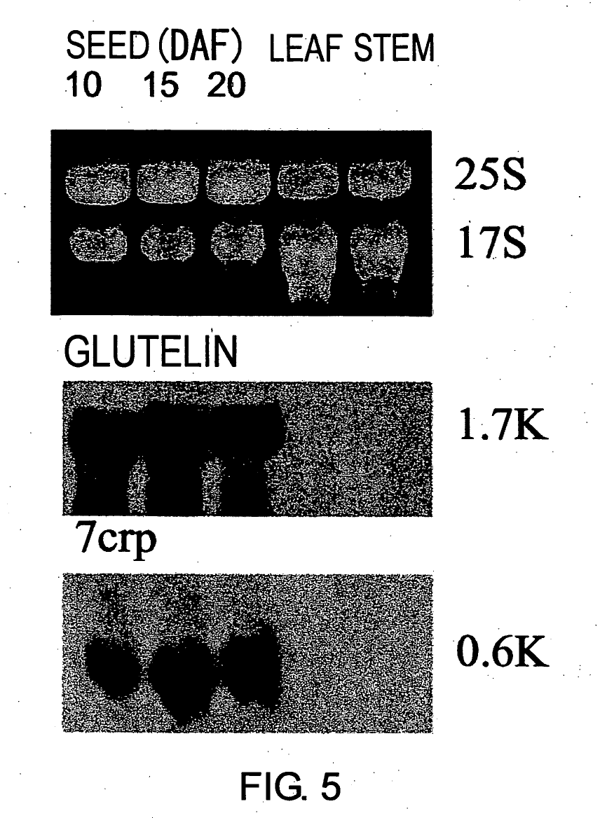

Similarly, expression pattern of the 7 Crp gene in seed at the grain-filling stage was examined by northern analysis. It was demonstrated that the expression level of 7 Crp mRNA reached a peak on the 15^{th} day after flowering and subsequently decreased. This expression pattern was extremely similar to that of the glutelin gene promoter used in the 7 Crp gene expression (FIG. 5). Also similarly as protein, the expression was not detected at mRNA level in other tissues.

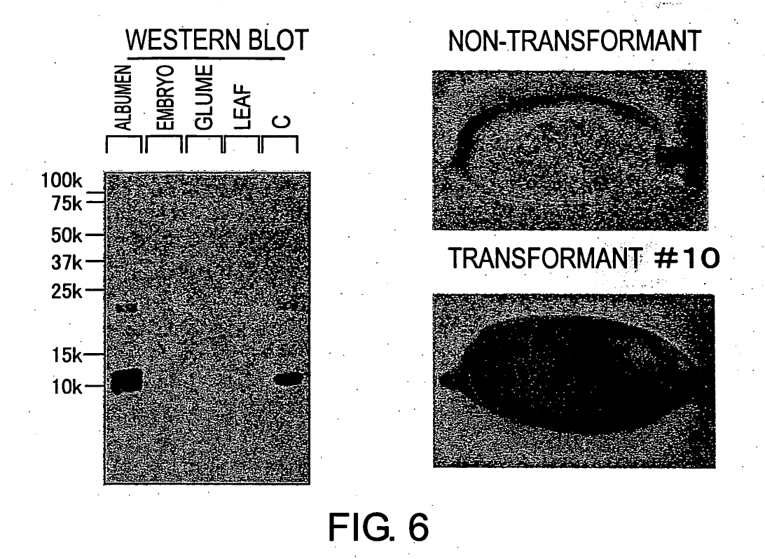

Next, in order to analyze the expression site of a T-cell epitope-linked peptide in seeds of the pGluBsig7CrpKDEL transformant #10, after seeds at the grain-filling stage were separated into embryo, albumen, and glume, proteins were extracted from the respective fractions, and subjected to western blot analysis. As a result, the T-cell epitope-linked peptide signal was detected only in albumen but not in samples extracted from embryo, glume, and leaf (FIG. 6). And, when a cross section of the transformant seed was prepared and subjected to tissue immunostaining using a specific antibody, T-cell epitope-linked peptide signal was also detected only in albumen, but not in embryo. From these results, it was confirmed that the T-cell epitope-linked peptide is specifically accumulated in albumen of the transformant seed. Since these results coincide with the expression site of the GluB-1 protein and that of a foreign protein using the GluB-1 promoter and signal sequence, it was proved that, also in the expression system of the T-cell epitope-linked peptide prepared in the present invention, the characteristic of the GluB-1 promoter is displayed.

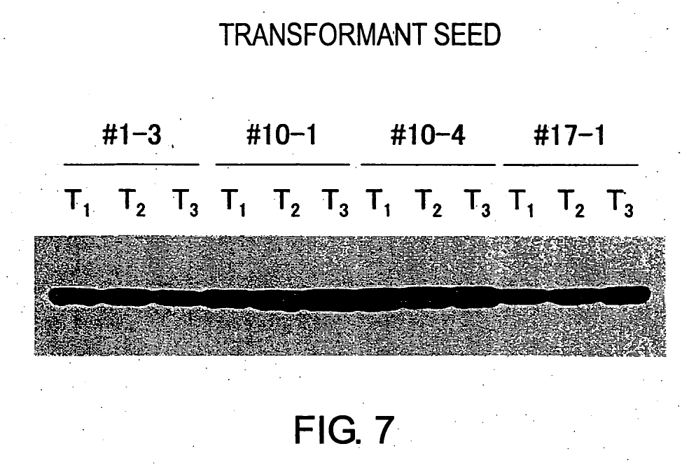

In addition, for examining changes in accumulation amounts of T-cell epitope-linked peptide in transformant seeds of advanced generations, T1, T2, and T3 fully ripened seeds were recovered from the respective pGluBsig7CrpKDEL transformant lines #1, #10, and #17, and western blot analysis was performed for proteins extracted from those seeds. As a result, with all the lines #1, #10, and #17, no changes in T-cell epitope-linked peptide signal were observed in T1, T2and T3 seeds. These results proved that, even in the advanced generation stages of the transformant, the T-cell epitope-linked peptide is produced in seeds with no changes in the accumulation amount thereof (FIG. 7).

### EXAMPLE 5

### Characteristic of a T-cell Epitope-Linked Peptide Product Expressed in Rice Seed

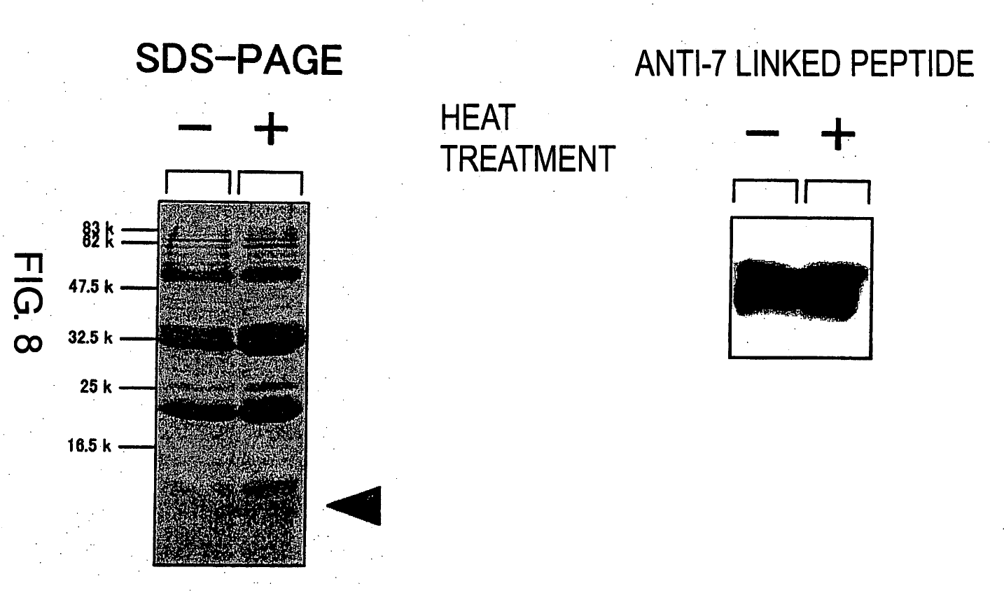

Using seeds of the pGluBsig7CrpKDEL transformant #10 accumulating the highest amount of T-cell epitope-linked peptide as an object, stability of T-cell epitope-linked peptide in seeds was examined when heat-treated in boiling water for 20 min. As a result of comparing T-cell epitope-linked peptide signals by western blot analysis, no changes were observed before and after the heat treatment (FIG. 8). From these results, it was determined that T-cell epitope-linked peptide accumulated in seeds remains stable even after cooking rice seeds in a rice cooker or the like.

In several allergenic proteins, it has been pointed out that a sugar chain bound to an allergen is a main cause in the induction of allergic reactions. In the case of a T-cell epitope-linked peptide derived from Japanese cedar pollen allergen, the research object of the present invention, since a typical N-type sugar chain-linked sequence hardly exists in its primary structure sequence, it is presumed that no sugar chain is bound. Therefore, for a T-cell epitope-linked peptide produced in rice seeds, an experiment was carried out to confirm this lack of sugar chain binding at the peptide level.

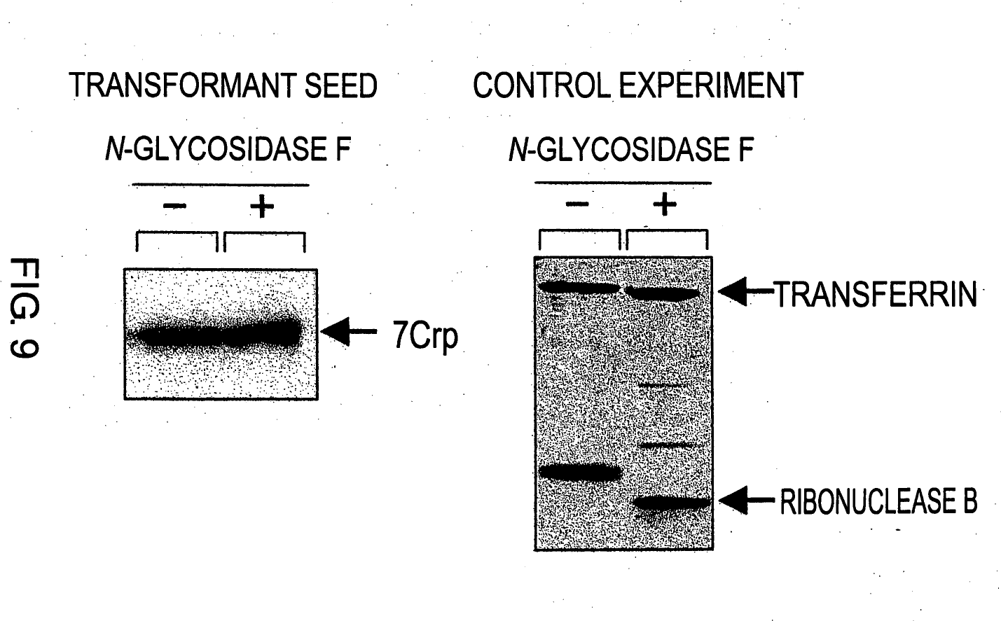

Endoglycosidase is an enzyme having the activity to act on the linkage of an N-linked sugar chain and release the sugar chain, and thus used in N-linked sugar chain analysis. When an N-linked sugar chain is present, the sugar chain is released from protein by endoglycosidase such that the molecular weight of protein is altered before and after the reaction. Therefore, the protein fraction containing the T-cell epitope-linked peptide was extracted from rice seeds and reacted with endoglycosidase to analyze the molecular weight of the T-cell epitope-linked peptide by western blot technique. As a result, no change in the molecular weight of the T-cell epitope-linked peptide was observed before and after the endoglycosidase reaction (FIG. 9). These results proved that there is no binding of an N-linked sugar chain to a T-cell epitope-linked peptide produced in rice seeds.

On the other hand, for identifying an N-terminal amino acid residue of a T-cell epitope-linked peptide accumulated in the pGluBsig7CrpKDEL transformant seeds, after seed proteins were extracted, separated by two-dimensional electrophoresis, and blotted onto PVDF membrane, the Tell epitope-linked peptide signal was detected with a specific antibody. As a result, the T-cell epitope-linked peptide behaved as a basic protein. This result coincides with the putative isoelectric point obtained from the amino acid sequence of the T-cell epitope-linked peptide being 9.76.

A spot of this T-cell epitope-linked peptide was recovered and the N-terminal amino acid sequence analysis thereof was requested. As a result, identification of the N-terminal amino acid residue was found to be difficult, suggesting that the N-terminal undergoes modification. Therefore, using the presently available reagents in the method for analyzing the modified terminal, specific enzymes to release the modification groups, N-formyl, N-pyroglutamyl, and N-acetyl groups respectively, the respective modification groups were removed, and the N-terminal amino acid sequence analysis was performed in the hope of identifying the N-terminal residue. However, identification of amino acid residue at the N-terminal of the T-cell epitope-linked peptide failed, suggesting that the N-terminal of the T-cell epitope-linked peptide in seeds is modified with groups other than formyl, pyroglutamyl, and acetyl groups.

### EXAMPLE 6

### Effect of Introduction of a T-cell Epitope-Linked Peptide Expression System on Rice Allergenic Protein

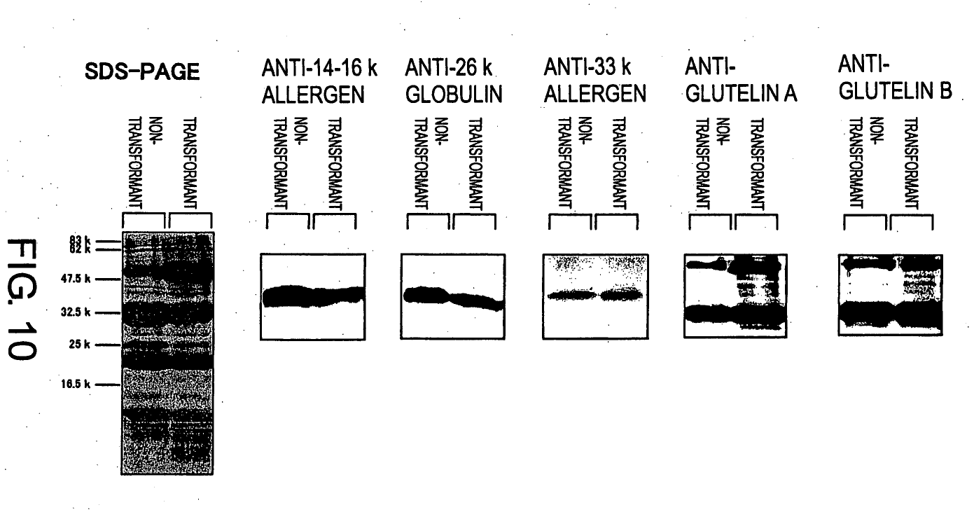

To examine the effects of the pGluBsig7CrpKDEL introduction on the main allergenic proteins of seeds, western blot analysis was performed using specific antibodies for these allergens. As a result, as compared to nontransformant seeds, in seeds of the pGluBsig7CrpKDEL transformant #10, an increase in the glutelin A precursor and decrease in the 14-16 k allergenic proteins was observed. At present, the reasons for these differences thus observed have not been elucidated. When antibodies against other allergens (26 k and 33 k globulins) were used, no changes such as those observed in the allergenic protein signals were confirmed (FIG. 10).

### EXAMPLE 7

### Accumulation of a T-cell Epitope-Linked Peptide by ADP Glucose Pyrophosphorylase Promoter

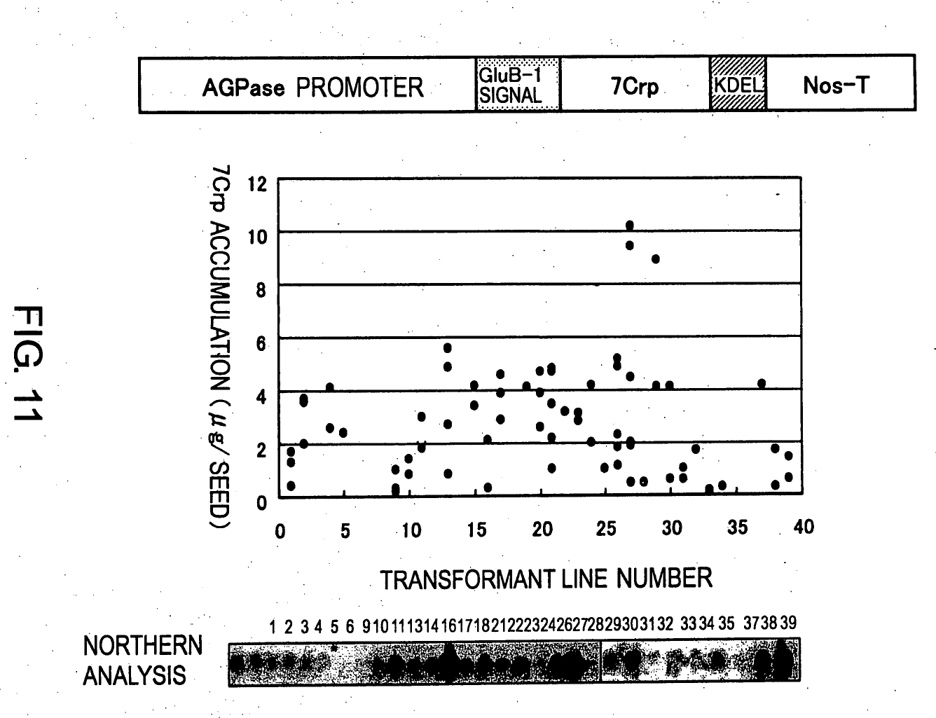

An expression plasmid for expressing a Japanese cedar pollen allergen T-cell epitope-linked peptide in rice seeds was prepared. The expression plasmid pAGPase sig7CrpKDEL was constructed by linking the promoter for ADP glucose pyrophosphorylase, a signal sequence, and a T-cell epitope peptide followed by adding KDEL and Nos-T sequences to the 3′-end thereof. The plasmid structure is shown in the upper panel of FIG. 11.

Next, when the expression pattern of the 7 Crp gene was examined, it was expressed in not only albumen of the seed but also embryo or vascular bundle with the highest expression level thereof being observed in the seed among plant organs. Accordingly, it was proved that the peptide can be accumulated in seeds even by using the ADP glucose pyrophosphorylase promoter.

Next, the amount of 7 Crp accumulated in fully ripened seeds of the transformant thus obtained was analyzed by western blot in a similar manner to Example 2. Quantification results of the 7 Crp accumulation amount are shown in the middle panel of FIG. 11. Although, as compared to the case where the 2.3 k GluB-1 promoter was used, 7 Crp accumulation was lowered in the amount, it was observed in many transformant lines. Further, the 7 Crp gene transcript in seeds at the grain-filling stage of T0 generation transformant was examined by northern analysis. Results are shown in the lower panel of FIG. 11.

From the above-described results, it was demonstrated that the accumulation method of the present invention can be performed even when promoters other than the glutelin promoter (capable of expressing the gene in albumen and embryo of seed or vascular bundle with the strongest expression in seed among plant organs) are used.

### EXAMPLE 8

### Accumulation of Storage Protein Comprising 2×7 Crp Inserted into a Variable Region Thereof

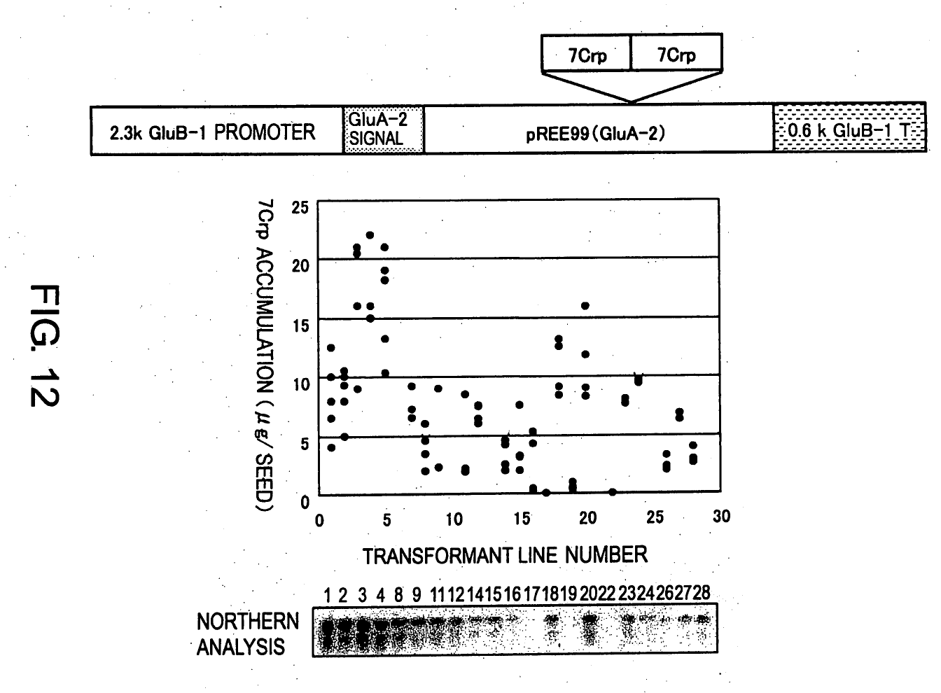

Into the variable region of the cDNA clone pREE99 of the glutelin GluA-2 -gene, two DNAs encoding the 7 Crp peptide were inserted in tandem to be expressed. In the upper panel of FIG. 12, the structure of the plasmid pREE99 2×7Crp is shown.

Next, the amount of 7 Crp accumulated in fully ripened seeds of the transformant thus obtained was quantified by western blot technique in a similar manner to Example 2. Two of the 7 Crp inserted into the variable region were accumulated as a part of glutelin. Results are shown in the middle panel of FIG. 12. Further, transcripts of the 7 Crp genes inserted into pREE99 was examined in seeds at the grain-filling stage of T0 generation transformant by northern analysis. Results are shown in the photograph in the lower panel of FIG. 12.

### EXAMPLE 9

### Comparison of T-cell Epitope Peptide Accumulation Level Due to Differences in Promoters

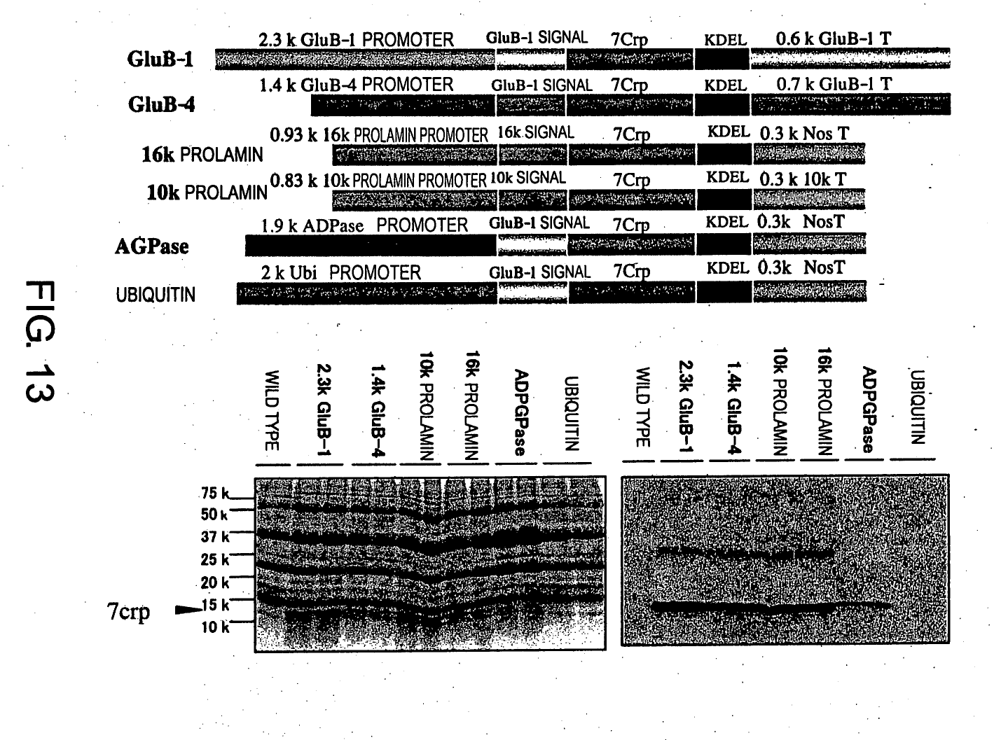

Using not only the glutelin GluB-1 promoter, whose high expression has been reported in the rice seed albumen, but also the glutelin GluB4, 10 kD and 16 kD prolamin promoters (respective promoters also contain the signal peptide), a T-cell epitope peptide was expressed in rice seeds. As the control, using the generally used constitutive promoters, such as the corn ubiquitin promoter and rice ADP glucose pyrophosphorylase (AGPase) promoter, the transformant rice expressing a T-cell epitope peptide was obtained (structures of genes having respective promoters are shown in the upper panel of FIG. 13). As for the respective gene constructs, independent 20 to 40 lines were produced, and lines showing the highest accumulation level in fully ripened seeds were compared.

With all the high expression albumen promoters used, 40 to 50 μg of T-cell epitope peptide per grain were accumulated. With the GluB-1 promoter, accumulation of the highest level 60 μg/grain was observed.

On the other hand, with the corn ubiquitin promoter and the AGPase promoter showing the constitutive expression, the accumulation level was 0.5 μg and 10 μg per grain even at the maximum, respectively.

Thus, it was proved that the use of an albumen-specific high expression promoter enables the accumulation of T-cell epitope peptide at high levels in seeds.

### EXAMPLE 10

### Comparison of T-cell Epitope Peptide Accumulation Level Due to Difference in Intracellular Localization Site

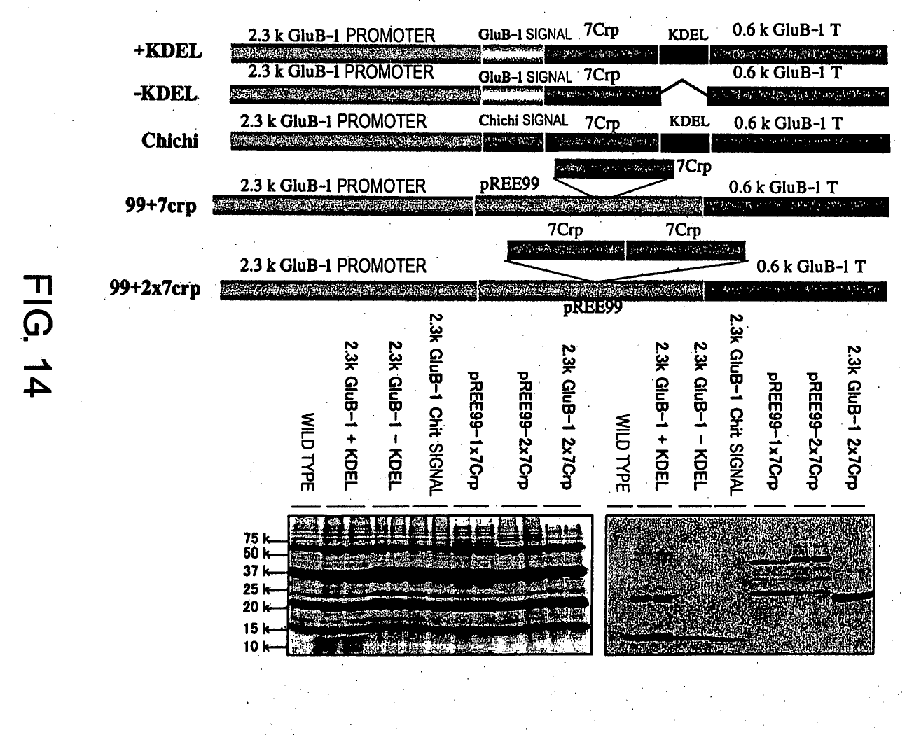

By adding the ER-retention signal KDEL so as to accumulate the T-cell epitope peptide in endoplasmic reticulum site, by adding the chitinase signal so as to positively transport the T-cell epitope peptide to the outside of cells to accumulate it into cell wall, or by inserting the peptide to the variable region of glutelin so as to accumulate the peptide as a part of glutelin in the protein-grain II, how the accumulation amount varies was examined. Structures of respective genes are shown in the upper panel of FIG. 14.

Addition of KDEL resulted in about 4-fold increase in the accumulation amount on the average level. By adding the chitinase signal, the accumulation level decreased to about ¼ compared to the case of KDEL addition, and the cell wall could store the T-cell epitope peptide but was not suitable as its accumulation site. The accumulation level of the peptide in the protein-grain II was about the same as that by the KDEL addition. When two T-cell epitope peptide genes were inserted in tandem into the variable region of glutelin acidic subunit gene, only the glutelin precursor was accumulated but the mature T-cell epitope peptide-inserted acidic subunit was not. These results were probably due to the inhibition of the precursor maturation by insertion of the T-cell epitope peptides or the degradation of the T-cell epitope peptide-inserted acidic subunit.

On the other hand, when two T-cell epitope peptide genes linked in tandem were directly expressed with the addition of KDEL, the accumulation level was elevated as compared to the case of a single T-cell epitope peptide gene addition.

### EXAMPLE 11

### Efficacy Assessment Test by Oral Administration of a T-cell Epitope Peptide-Accumulated Rice to Mouse

Japanese cedar allergen Cry j1 (1 μg) and alum (10 μg) per mouse (B10S) were intranasally administered every other day 9 times, and subsequently mice were fed with the rice powder containing 516 μg of T-cell epitope peptide (7 Crp) mixed with feed for 31 days. Further, after Cry j1 (1 μg) and alum (10 μg) were intranasally administered every other day 3 times, the mice were dissected one week later, and the spleen was taken out to measure the T-cell proliferation potency and IgE antibody titer. The experiment was performed using male mice.

In the T-cell epitope assessment method, when lymph node cells collected from the immunized mice were stimulated with Cry j1 or the p1-211-225 epitope of Cry j1 in vitro, it was assessed whether the lymph node cells showed the proliferative reaction to these stimulations using the intracellular uptake value of [³H]thymidine as an indicator.

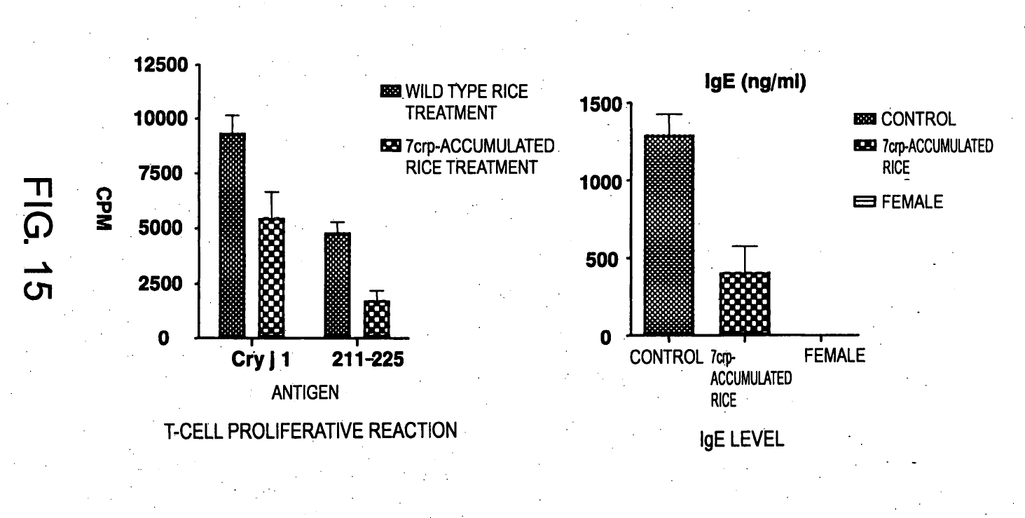

As a result of these experiments, the proliferation potency of T-cells specifically recognizing the 211-225 epitope of Cry j1 was found to be lowered to 70% level as compared to the mouse fed with non-recombinant rice (FIG. 15, left). In addition, the Cry j1 allergen-specific IgE antibody titer was also decreased to about ⅓ (FIG. 15, right). These results demonstrate that it is possible to induce immune tolerance by feeding an animal rice accumulated with a T-cell epitope peptide (7 Crp) and, thereby, mitigate pollinosis.

### INDUSTRIAL APPLICABILITY

By the present invention, a T-cell epitope-linked peptide having effect to mitigate (treat) Japanese cedar pollinosis was successfully produced in rice seeds. By the result of the present invention, it becomes possible to produce a T-cell epitope-linked peptide more inexpensively than the current T-cell epitope-linked peptide production system using chemical synthetic method or synthesis in E. coli.

The T-cell epitope peptide accumulated in seeds is extremely stable even when stored at room temperature (for one year or more). The yield thereof is also easily controlled. And its production requires no special facility but only a farm field. Further, its oral intake through the daily diet enables to cut down the cost and medical expenses necessary for the conventional administration such as subcutaneous injection so that it becomes possible to administer the T-cell epitope-linked peptide at a lower cost. By making good use of the rice seed production system having these advantages, there can be expected the creation of new business for production and supply of medically useful ingredients such as vaccines against allergic diseases and peptides for mitigating lifestyle-related diseases at a lower cost.

1. A DNA comprising a structure in which any one of DNA (a), DNA (b), or DNA (c) is placed under the control of a storage protein promoter, wherein
DNA (a) comprises a DNA in which a DNA encoding a storage protein signal sequence is added to the 5′-end of a DNA encoding an allergen-specific T-cell epitope peptide and/or a DNA encoding an ER-retention signal sequence is added to the 3′-end thereof;
DNA (b) comprises a DNA encoding a polypeptide in which a storage protein signal sequence is added to the N-terminal of an allergen-specific T-cell epitope peptide and/or an ER-retention signal sequence is added to the C-terminal thereof; and
DNA (c) comprises a DNA encoding a polypeptide having a structure in which an allergen-specific T-cell epitope peptide is inserted into a variable region of a storage protein.

2. A vector for preparing a plant accumulating a T-cell epitope, wherein said vector comprises the DNA according to claim 1.

3. A host cell comprising the DNA according to claim 1.

4. A method for accumulating an allergen-specific T-cell epitope in a plant, wherein said method comprises the step of introducing the DNA according to claim 1 into a plant.

5. A method for accumulating a T-cell epitope in a plant, wherein said method comprises the steps of:
(a) obtaining a DNA encoding an allergen-specific T-cell epitope peptide;
(b) adding a DNA encoding a storage protein signal sequence to the 5′-end of the DNA obtained in (a), and/or a DNA encoding an ER-retention signal sequence to the 3′-end thereof; and
(c) expressing the DNA of (b) under the control of a storage protein promoter in a plant.

6. A method for accumulating a T-cell epitope in a plant, wherein said method comprises the steps of:
(a) obtaining a DNA encoding an allergen-specific T-cell epitope peptide; and
(b) inserting the DNA of (a) into a DNA region encoding a variable region of a plant storage protein to express the DNA.

7. The method according to claim 4, wherein said allergen is a Japanese cedar pollen allergen.

8. The method according to claim 7, wherein said Japanese cedar pollen allergen is Cry j1 and Cry j2.

9. The method according to claim 4, wherein said T-cell epitope is accumulated in an edible part of a plant.

10. The method according to claim 9, wherein said edible part is a seed.

11. A transgenic plant produced by the method according to claim 4, wherein said plant comprises a T-cell epitope accumulated therein.

12. A transgenic plant which is a progeny or a clone of the plant according to claim 11.

13. A cell derived from the plant according to claim 11.

14. A breeding material of the plant according to claim 11.

15. A seed of the plant according to claim 11.

16. The seed according to claim 15, wherein said seed is thermostable.

17. The transgenic plant according to claim 11, wherein said plant comprises rice having a T-cell epitope accumulated therein.

18. A food composition for treating or preventing an allergic disease, wherein said food composition comprises the seed according to claim 15 as an effective ingredient.

19. The food composition according to claim 18, wherein said allergic disease is a type I allergy.

20. A method for producing a transgenic plant comprising a T-cell epitope accumulated therein using the method according to claim 4.

21. A method of producing a rice comprising a T-cell epitope accumulated therein using the method according to claim 10.

22. A rice comprising an allergen-specific T-cell epitope accumulated in albumen.

23. A food/drink product comprising the rice according to claim 22, wherein said product has an activity associated with the prevention, treatment, or alleviation of an allergic disease.

24. The rice according to claim 22, wherein said allergen is a pollen allergen.

25. A food/drink product comprising the rice according to claim 24, wherein said product has an activity associated with the prevention, treatment, or alleviation of pollinosis.

26. The method according to claim 5, wherein said allergen is a Japanese cedar pollen allergen.

27. The method according to claim 6, wherein said allergen is a Japanese cedar pollen allergen.

28. The method according to claim 26, wherein said Japanese cedar pollen allergen is Cry j1 and Cry j2.

29. The method according to claim 27, wherein said Japanese cedar pollen allergen is Cry j1 and Cry j2.

30. The method according to claim 5, wherein said T-cell epitope is accumulated in an edible part of a plant.

31. The method according to claim 6, wherein said T-cell epitope is accumulated in an edible part of a plant.

32. The method according to claim 30, wherein said edible part is a seed.

33. The method according to claim 31, wherein said edible part is a seed.

34. A cell derived from the plant according to claim 12.

35. A breeding material of the plant according to claim 12.

36. A seed of the plant according to claim 12.

37. The seed according to claim 36, wherein said seed is thermostable.

38. A food composition for treating or preventing an allergic disease, wherein said food composition comprises the seed according to claim 16 as an effective ingredient.

39. A food composition for treating or preventing an allergic disease, wherein said food composition comprises the seed according to claim 17 as an effective ingredient.

40. A host cell comprising the vector according to claim 2.

41. A method for accumulating an allergen-specific T-cell epitope in a plant, wherein said method comprises the step of introducing the vector according to claim 2 into a plant.

42. A transgenic plant produced by the method according to claim 5, wherein said plant comprises a T-cell epitope accumulated therein.

43. A transgenic plant produced by the method according to claim 6, wherein said plant comprises a T-cell epitope accumulated therein.

44. A method for producing a transgenic plant comprising a T-cell epitope accumulated therein using the method according to claim 5.

45. A method for producing a transgenic plant comprising a T-cell epitope accumulated therein using the method according to claim 6.
